(19) United States
(12) Patent Application Publication
(10) Pub. No.: US 2025/0352790 A1
(43) Pub. Date: Nov. 20, 2025

(54) DEVICE RELATED TO THERAPEUTIC DNA DELIVERY

(71) Applicant: RenBio, Inc., Long Island City, NY (US)

(72) Inventors: Rachel LIBERATORE, Long Island City, NY (US); Neal PADTE, Long Island City, NY (US); Chasity ANDREWS, Long Island City, NY (US); Andrew CAMERON, Long Island City, NY (US); Debnath MAJI, Long Island City, NY (US)

(21) Appl. No.: 18/862,772

(22) PCT Filed: May 5, 2023
(86) PCT No.: PCT/US2023/066671

§ 371 Date: Nov. 4, 2024

Related U.S. Application Data
(60) Provisional application No. 63/338,753, filed on May 5, 2022.
(60) Provisional application No. 63/338,774, filed on May 5, 2022.

(51) Int. Cl. A61N 1/32; A61K 9/00; A61K 31/711; A61K 48/00; A61N 1/05

(52) U.S. Cl. CPC A61N 1/327; A61K 9/0019; A61K 31/711; A61K 48/005; A61N 1/0502

(57) ABSTRACT

The present disclosure relates to, in part, devices that allow for the delivery of an antibody or a therapeutic protein, or a fragment thereof, in vivo, and the devices are useful for treatment of cancer, inflammatory diseases, and infectious diseases.

### PRIORITY

This application claims the benefit of, and claims priority to, U.S. Provisional Application No. 63/338,753, filed May 5, 2022, and U.S. Provisional Application No. 63/338,774, filed May 5, 2022 which are each hereby incorporated by reference in their entireties.

### DESCRIPTION OF THE TEXT FILE SUBMITTED ELECTRONICALLY

The instant application contains a sequence listing, which has been submitted in XML format via EFS-Web. The contents of the XML copy named “RBF-001PC_SEQUENCE_LISTING,” which was created on May 5, 2023 and is 470,000 bytes in size, the contents of which are incorporated herein by reference in their entirety.

### FIELD OF THE DISCLOSURE

The present disclosure relates to, in part, devices that allow for the delivery of an antibody or a therapeutic protein, or a fragment thereof, in vivo, and the devices are useful for treatment of cancer, inflammatory diseases, and infectious diseases.

### BACKGROUND

Cancer, inflammatory diseases, and various infectious diseases, including rare diseases, are significant health problems worldwide, taking millions of lives each year and thus taking an enormous toll on human resources and the economy. Despite advances that have been made in detection and therapy of cancer and various infection diseases, no vaccine or other universally successful method for prevention or treatment is currently available. For example, treatment of infectious diseases, although generally more advanced and managed using preventative vaccines in many cases, faces issues such as strain diversity and appearance of new strains, including those carrying (multi) antibiotic resistance. Increased clinical development and use of therapeutic proteins, including monoclonal antibodies, have made significant positive impacts on patient health in many disease areas, but they are expensive drugs to make and the administration methods and frequency are burdensome to clinicians and patients and limit their more widespread use.

Plasmid transfer technology for use in cancer, inflammatory diseases, and various infectious diseases has traditionally been limited in scope because in vivo expression levels resulting from the naked DNA transfer have been low, and for example, viral vectors are typically immunogenic, and thus, the immune response generated against the viral vector from a first administration prevents efficient redosing.

Thus, there is a need for effective and targeted plasmid transfer devices to treat various cancers and other diseases.

### SUMMARY

Accordingly, in various aspects, the present disclosure relates to a gene transfer device, the device comprising a handpiece; an array of electrodes arranged at one end of the handpiece and configured to be positioned at a host cell of a subject; and a pulse generator configured to generate electric pulses that cause the array of electrodes to emit electric fields in the targeted tissue to maximize expression of a plasmid DNA construct delivered therethrough while minimizing applied voltage and total electrical dose. The direct administration of plasmid DNA encoding therapeutic proteins and monoclonal antibodies using the gene transfer device disclosed herein significantly increase the impact of drugs by reducing both the cost and dosing frequency.

In some embodiments, disclosed herein is a device for gene transfer, the device comprising: a handpiece; an array of electrodes arranged at one end of the handpiece and configured to be positioned at a host cell of a subject; and a pulse generator configured to generate electric pulses that cause the array of electrodes to emit electric fields in the targeted tissue to maximize expression of a plasmid DNA construct delivered therethrough while minimizing applied voltage and total electrical dose.

In some embodiments, the device further comprises a DNA injection port configured to administer the plasmid DNA to the host cell, wherein the electrode array comprises a first electrode, a second electrode, a third electrode, a fourth electrode, a fifth electrode and a sixth electrode positioned circumferentially around the DNA injection port.

In some embodiments, a respective electric pulse generated by the pulse generator travels vertically or diagonally from the first electrode to at least one of the third, fourth, and/or fifth electrodes.

In some embodiments, a respective electric pulse generated by the pulse generator travels vertically or diagonally from the first electrode to at least two of the third, fourth, and/or fifth electrodes.

In some embodiments, a respective electric pulse generated by the pulse generator travels vertically or diagonally from the second electrode to at least one of the fourth, fifth, and/or sixth electrodes.

In some embodiments, a respective electric pulse generated by the pulse generator travels vertically or diagonally from the second electrode to at least two of the fourth, fifth and/or sixth electrodes.

In some embodiments, a respective electric pulse generated by the pulse generator travels vertically or diagonally from the third electrode to at least one of the first, fifth, and/or sixth electrodes.

In some embodiments, a respective electric pulse generated by the pulse generator travels vertically or diagonally from the third electrode to at least two of the first, fifth and/or sixth electrodes.

In some embodiments, a respective electric pulse generated by the pulse generator travels vertically or diagonally from the fourth electrode to at least one of the first, sixth, and/or second electrodes.

In some embodiments, a respective electric pulse generated by the pulse generator travels vertically or diagonally from the fourth electrode to at least two of the first, fifth and/or sixth electrodes.

In some embodiments, a respective electric pulse generated by the pulse generator travels vertically or diagonally from the fifth electrode to at least one of the first, second, and/or third electrodes.

In some embodiments, a respective electric pulse generated by the pulse generator travels vertically or diagonally from the fifth electrode to at least two of the first, second, and/or third electrodes.

In some embodiments, a respective electric pulse generated by the pulse generator travels vertically or diagonally from the sixth electrode to at least one of the second, third, and/or fourth electrodes.

In some embodiments, a respective electric pulse generated by the pulse generator travels vertically or diagonally from the sixth electrode to at least two of the second, third, and/or fourth electrodes.

In some embodiments, a respective electric pulse generated by the pulse generator travels from the fourth electrode to at least one of the first electrode and the second electrode.

In some embodiments, a respective electric pulse generated by the pulse generator travels vertically from the fourth electrode to the second electrode.

In some embodiments, the electrode array comprises 6 electrodes and a single DNA injection port. In some embodiments, the electrodes are positioned according to a pattern as shown in FIG. 1A, FIG. 1B, FIG. 2A, FIG. 2B, FIG. 2C, FIG. 2D, FIG. 2E, FIG. 2F, FIG. 2G, and FIG. 2H. In some embodiments, the electrodes are positioned according to FIG. 1A and FIG. 1B.

In some embodiments, a respective electric pulse generated by the pulse generator travels diagonally from the fourth electrode to the first electrode.

In some embodiments, a respective electric pulse generated by the pulse generator travels from the fifth electrode to at least one of the first electrode and the second electrode.

In some embodiments, a respective electric pulse generated by the pulse generator travels from the fifth electrode to the first electrode.

In some embodiments, a respective electric pulse generated by the pulse generator travels diagonally from the fifth electrode to the second electrode.

In some embodiments, a respective electric pulse generated by the pulse generator travels horizontally from the sixth electrode to the third electrode.

In some embodiments, the pulse is a perpendicular pulse relative to the orientation of a muscle fiber.

In some embodiments, the pulse is a parallel pulse relative to the orientation of a muscle fiber.

In some embodiments, the device comprises an injection needle tip and an electrode needle tip having a distance of one of: at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, or at least 20 mm between the injection needle tip and the electrode needle tip.

In some embodiments, the electric pulses have a pulse pattern in the range of 1 MHz to 1,000 KHz.

In some embodiments, the pulse pattern has at least 100, or at least 200, or at least 300, or at least 400, or at least 500, or at least 1000, or at least 2,500, or at least 5000 pulses, or at least 10,000 pulses, or at least 20,000 pulses, or at least 50,000 pulses for each burst.

In some embodiments, electrode 4 pulses to at least one of electrode 1 and electrode 2. In some embodiments, electrode 4 pulses up to electrode 2. In some embodiments, electrode 4 pulses diagonally to electrode 1. In some embodiments, electrode 5 pulses up to at least one of electrode 1 and electrode 2. In some embodiments, electrode 5 pulses up to electrode 1. In some embodiments, electrode 5 pulses diagonally to electrode 2. In some embodiments, electrode 6 pulses horizontally to electrode 3. In some embodiments, the pulse is a perpendicular pulse relative to the orientation of a muscle fiber. In some embodiments, the pulse is a parallel pulse relative to the orientation of a muscle fiber. In some embodiments, the device comprises an injection needle tip and an electrode needle tip having a distance of at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, or at least 20 mm between the injection needle tip and the electrode needle tip. In some embodiments, disclosed herein is a method of delivering DNA to a subject, the method comprising:
a. loading the device of any one of the preceding embodiments with a plasmid DNA construct encoding an antibody or a therapeutic protein; and
b. injecting the DNA plasmid into a host cell, thereby delivering the DNA to the subject.
   In some embodiments, the host cell is a muscle cell. In some embodiments, the DNA is injected both intramuscularly and in the extracellular space of the host cell. In some embodiments, the DNA is taken up in the host cell by electroporation. In some embodiments, the plasmid DNA construct is selected from SEQ ID NOs: 1-27.

In some embodiments, a method for treating or preventing cancer is disclosed herein based on the gene transfer device. In some embodiments, the method comprises administering an effective amount of a DNA composition, such as a plasmid DNA construct, to a tissue site of a patient in need thereof, based on the gene transfer device disclosed herein. In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient who has a cancer, such as a solid tumor or a blood cancer. In some embodiments, the cancer is selected form one or more of a cancer of a blood vessel, an eye tumor, basal cell carcinoma, biliary tract cancer; bladder cancer; bone cancer; brain and central nervous system cancer; primary breast cancer; metastatic breast cancer colorectal cancer, cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma (e.g., Kaposi's sarcoma); skin cancer; squamous cell cancer; stomach cancer; testicular cancer; thyroid cancer; uterine or endometrial cancer; cancer of the urinary system; vulvar cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs syndrome.

In some embodiments, a method for treating or preventing an inflammatory or autoimmune disease or disorder is disclosed herein based on the gene transfer device. In some embodiments, the method comprises administering an effective amount of a DNA composition, such as a plasmid DNA construct, to a tissue site of a patient in need thereof, based on the gene transfer device disclosed herein. In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient who has an inflammatory or autoimmune disease or disorder. In some embodiments, the autoimmune disease or disorder is selected from graft versus host disease, transplantation rejection (e.g., prevention of allograft rejection), multiple sclerosis, diabetes mellitus, lupus, celiac disease, Crohn's disease, pediatric Crohn's disease, ulcerative colitis, Guillain-Barre syndrome, scleroderma, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Meniere's syndrome; pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, rheumatoid arthritis, psoriatic arthritis, plaque psoriasis, ankylosing spondylitis, and juvenile idiopathic arthritis.

In some embodiments, a method for treating or preventing an inflammatory eye disease is disclosed herein based on the gene transfer device. In some embodiments, the method comprises administering an effective amount of a DNA composition, such as a plasmid DNA construct, to a tissue site of a patient in need thereof, based on the gene transfer device disclosed herein. In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient who has an inflammatory eye disease selected from an inflammatory eye disease associated with corneal transplant, diabetic macular edema, diabetic retinopathy, dry eye disease, scleritis, blepharitis, keratitis, conjunctivitis, chorioretinal inflammation, chorioretinitis, iridocyclitis, iritis, posterior cyclitis, and uveitis.

In some embodiments, a method for improving a patient response to allogeneic hematopoietic stem cell transplantation (aHSCT) is disclosed herein based on the gene transfer device. In some embodiments, the method comprises administering an effective amount of a DNA composition, such as a plasmid DNA construct, to a tissue site of a patient in need thereof, based on the gene transfer device disclosed herein. In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient for improving the patient's response to allogeneic hematopoietic stem cell transplantation (aHSCT).

In some embodiments, a method for treating or preventing a rare disease is disclosed herein based on the gene transfer device. In some embodiments, the method comprises administering an effective amount of a DNA composition, such as a plasmid DNA construct, to a tissue site of a patient in need thereof, based on the gene transfer device disclosed herein. In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient who has a rare disease selected from severe chronic neutropenia, WHIM Syndrome, Aminoacylase 1 deficiency, Apo A-I deficiency, Carbamoyl phosphate synthetase 1 deficiency, Omithine transcarbamylase deficiency, Plasminogen activator inhibitor type 1 deficiency, Flaujeac factor deficiency, High-molecular-weight kininogen deficiency congenital, PEPCK 1 deficiency, Pyruvate kinase deficiency liver type, Alpha 1-antitrypsin deficiency, Anti-plasmin deficiency congenital, Apolipoprotein C 21 deficiency, Butyrylcholinesterase deficiency, Complement component 2 deficiency, Complement component 8 deficiency type 2, Congenital antithrombin deficiency type 1, Congenital antithrombin deficiency type 2, Congenital antithrombin deficiency type 3, Cortisone reductase deficiency 1, Factor VII deficiency, Factor X deficiency, Factor XI deficiency, Factor XII deficiency, Factor XIII deficiency, Fibrinogen deficiency congenital, Fructose-1 6-bisphosphatase deficiency, Gamma aminobutyric acid transaminase deficiency, Gamma-cystathionase deficiency, Glut2 deficiency, GTP cyclohydrolase I deficiency, Isolated growth hormone deficiency type 1B, Molybdenum cofactor deficiency, Prekallikrein deficiency congenital, Proconvertin deficiency congenital, Protein S deficiency, Pseudocholinesterase deficiency, Stuart factor deficiency congenital, Tetrahydrobiopterin deficiency, Type 1 plasminogen deficiency, Urocanase deficiency, Chondrodysplasia punctata with steroid sulfatase deficiency, Homocystinuria due to CBS deficiency, Guanidinoacetate methyltransferase deficiency, Pulmonary surfactant protein B deficiency, Acid Sphingomyelinase Deficiency, Adenylosuccinate Lyase Deficiency, Aggressive Angiomyxoma, Albrights Hereditary Osteodystrophy, Carney Stratakis Syndrome, Carney Triad Syndrome, CDKL5 Mutation, CLOVES Syndrome, Cockayne Syndrome, Congenital

Disorder of Glycosylation type IR, Cowden Syndrome, DEND Syndrome, Dercum's Disease, Febrile Infection-Related Epilepsy Syndrome, Fibular Aplasia Tibial Campomelia Oligosyndactyly Syndrome, Food Protein-Induced Enterocolitis Syndrome, Foreign Body Giant Cell Reactive Tissue Disease, Galloway-Mowat, Gitelman syndrome, Glycerol Kinase Deficiency, Glycogen Storage Disease type 9, gml gangliosidosis, Hereditary spherocytosis, Hidradenitis Suppurativa Stage III, Horizonatal Gaze Palsy with Progressive Scoliosis, IMAGe syndrome, Isodicentric chromosome 15, isolated hemihyperplasia, Juvenile Xanthogranuloma, Kasabach-Merritt Syndrome, Kniest Dysplasia, Koolen de-Vries Syndrome, Lennox-Gastaut syndrome, Lymphangiomatosis, Lymphangiomiomytosis, MASA Syndrome, Mast Cell Activation disorder, Mecp2 Duplication Syndrome, Mucha Habermann, Neonatal Hemochromatosis, N-glycanase deficiency, Opsoclonus Myoclonus Syndrome, Persistent genital arousal disorder, Pompe Disease, Progressive Familial Intrahepatic Cholestasis, Pseudohypoparathyroidism type 1a, PTEN Hamartoma Tumor Syndrome, Schnitzler syndrome, Scleroderma, Semi Lobar Holoprosencephany, Sjogren's Syndrome, Specific Antibody Deficiency Disease, SYNGAP 1 deficiency, Trigeminal Trophic Syndrome, Undifferentiated Connective Tissue Disease, or X-linked hypophosphatemia.

In some embodiments, a method for treating or preventing a viral infection is disclosed herein based on the gene transfer device. In some embodiments, the method comprises administering an effective amount of a DNA composition, such as a plasmid DNA construct, to a tissue site of a patient in need thereof, based on the gene transfer device disclosed herein, In some embodiments, the administering includes at least one of electroporation and injection based on the gene transfer device disclosed herein. In some embodiments, the administering is intramuscular injection. In some embodiments, the administering comprises applying a stimulus to a muscle cell in the patient. In some embodiments, the stimulus is an electrical pulse. In some embodiments, the antibody or the therapeutic protein is expressed in the muscle cell. In some embodiments, the method further comprises detecting the antibody or the therapeutic protein in the patient's blood. In some embodiments, the administering increases the uptake of the antibody or the therapeutic protein in the patient's blood. In some embodiments, the gene transfer device is applied (e.g., by intramuscular injection) to a patient who has a viral infection wherein the virus is of the family Arbovirus, Arenaviridae, Arterivirus, Astroviridae, Bimaviridae, Bromo viridae, Bunyaviridae, Caliciviridae, Circoviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Papovaviridae, Parvoviridae, Paramyxoviridae, Picobirnaviridae, Picobimavirus, Picornaviridae, Poty viridae, Poxviridae, Reoviridae, Retroviridae, Roniviridae, Sequiviridae, Tenuivirus, Togaviridae, Tombusviridae, Totiviridae, or Tymoviridae. In some embodiments, the viral infection is selected from Alfuy virus, Banzi virus, bovine diarrhea virus, Chikungunya virus, Dengue virus (DNV), Epstein Barr Virus (EBV), Hepatitis B virus (HBV), Hepatitis C virus (HCV), herpes simplex virus type 1 (HSV-1), herpes simplex virus type 2 (HSV-2), human cytomegalovirus (hCMV), human immunodeficiency virus (HIV), Ilheus virus, influenza virus (including avian and swine isolates), rhinovirus, norovirus, adenovirus, Japanese encephalitis virus, Kaposi's sarcoma associated herpesvirus (KSHV), Kokobera virus, Kunjin virus, Kyasanur forest disease virus, louping-ill virus, measles virus, MERS-coronavirus (MERS), metapneumovirus, any of the Mosaic Viruses, Murray Valley virus, parainfluenza virus, poliovirus, Powassan virus, respiratory syncytial virus (RSV), Rocio virus, SARS-coronavirus (SARS), St. Louis encephalitis virus, tick-home encephalitis vims, West Nile virus (WNV), Ebola virus, Nipah virus, Lassa vims, Tacaribe virus, Junin vims, yellow fever vims, Varicella zoster virus (VZV), or vesicular stomatitis virus.

The details of one or more examples of the disclosure are set forth in the description below. Other features or advantages of the present disclosure will be apparent from the following drawings, detailed description of several examples, and also from the appended claims. The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A and FIG. 1B are images showing the electrode and injection configuration of the gene transfer device, also referred to as the “barrel device” herein. The solid dark and solid green circles represent an electrode, and the clear circle represents a DNA injection port. Red arrows indicate the pulsing direction.

FIG. 2A, FIG. 2B, FIG. 2C, FIG. 2D, FIG. 2E, FIG. 2F, FIG. 2G, and FIG. 2H are images showing the configuration of electrode pulsing patterns. Red arrows indicate the pulsing direction.

FIG. 3A, FIG. 3B, and FIG. 3C are images showing the configuration of electrode arrays and pulsing patterns. The solid dark and solid green circles represent an electrode, and the clear circle represents a DNA injection port. The dashed line clear circle represents an electrode with a DNA injection site at a side port. FIG. 3A (“4n device”) shows a device with four electrodes, FIG. 3B (“6n device”) shows a device with 6 electrodes, and FIG. 3C shows a device with electrodes in a star-type configuration (“star device”).

FIG. 4A, FIG. 4B, and FIG. 4C are images and graphs of the gene transfer device showing enhanced gene expression based on the configuration of the 6n device.

FIG. 5 are images and graphs of the gene transfer device showing enhanced DNA distribution and gene expression based on the barrel device and the configuration of electrode pulsing patterns in FIG. 1A and FIG. 1B.

FIG. 6 is a graph showing the barrel device increasing the level of IgG1 in rabbits over a time course of several weeks.

FIG. 7 is a graph showing the barrel device and 6n device increasing the level of human anti-influenza antibody levels in pigs.

FIG. 8 is an image showing a first-in-human (FIH) generator (left) and handpiece (right).

FIG. 9 shows images of the gene transfer devices disclosed herein. The top left image represents the orientation of a device relative to the fiber direction of a muscle. The bottom left image represents the tissue sectioning planes (used for imaging) as well as the directional axis used. The right half image represent four (4) devices and their respective electrode layout and pulsing scheme. The various shades on barrel indicate novel pairings/pulses of the electrodes.

FIG. 10 shows images demonstrating perpendicular pulsing affects more muscle fibers.

FIG. 11 shows graphs demonstrating how orientation affect electroporation efficiency.

FIG. 12 shows images of a 2D and 3D representation collected and evaluated for the localization of the injected fluid containing the plasmid DNA.

FIG. 13 is an image showing the expression pattern and yield is unique to each muscle.

FIG. 14 is an image showing tissue areas receiving sufficient electrical field are revealed by fluorophore expression.

FIG. 15 is an image showing the perpendicular pulse targets the center of the device footprint, while the parallel pulse expands the reach along the fibers.

FIG. 16 is a graph and image showing how the side-port edge effect spikes the current locally.

FIG. 17 is an image of the barrel device design configuration.

FIG. 18 shows images of COMSOL modeling to illustrate the contribution of each barrel pulse.

FIG. 19 are graphs demonstrating quantification of tdTomato showing the need for each pulse for maximal expression in barrel.

FIG. 20 is an image showing parallel vs perpendicular pulsing and the threshold for electroporation, and the how barrel device disclosed herein requires a lower electric field (V/cm) to successfully electroporate a target site.

FIG. 21 is an image showing PNA labeling and fluorescent imaging. The left panel of FIG. 21 shows an injection at 10 seconds, and the right panel of FIG. 21 shows an injection at 60 seconds.

FIG. 22 is an image showing PNA labeling and fluorescent imaging. The left panel of FIG. 22 shows an injection at 10 seconds, and the right panel of FIG. 22 shows an injection at 60 seconds.

FIG. 23A, FIG. 23B, and FIG. 23C are graphs (FIG. 23A) and images (FIG. 23B and FIG. 23C) showing the intensity, spread, and area of different experimental injection parameters.

FIG. 24 are graphs showing the post-injection wait in the vastus and biceps of the rat and the effect of electroporation to enhance protein expression in the target tissue.

FIG. 25 are graphs showing the effect of electroporation and different solutions used to enhance protein expression in the target tissue.

FIG. 26 is a graph showing the effect of electroporation and different solutions used to enhance protein expression in the target tissue.

FIG. 27 is a graph showing the effect of electroporation and different solutions used to enhance protein expression in the target tissue.

FIG. 28 is a graph showing the effect of electroporation and a solution with EDTA to enhance protein expression in the target tissue.

FIG. 29 is a graph showing the effect of electroporation and a solution with EDTA to enhance protein expression in the target tissue.

FIG. 30 are graphs showing the effect of electroporation and the addition of insulation to the electrodes to enhance protein expression in the target tissue.

FIG. 31 are graphs showing the effect of electroporation and different solutions used to enhance protein expression in the target tissue.

FIG. 32 are graphs showing the effect of voltage escalation to enhance protein expression in the target tissue.

FIG. 33 is an image showing impedance spectroscopy of a target muscle.

FIG. 34A, FIG. 34B, FIG. 34C, FIG. 34D, and FIG. 34E are graphs and images showing impedance curve fitting and extracting parameters in a electrical circuit model.

### DETAILED DESCRIPTION

The present invention is based, in part, on the surprising discovery of gene transfer devices that delivers a plasmid DNA construct through short electrical pulses. The gene transfer devices disclosed herein are designed to feature an electrode array design and electrical pulsing parameters that are suggested to be suboptimal by published literature. Further, the gene transfer devices disclosed herein are shown to deliver the plasmid DNA construct to the muscle cell, which allowed for the simultaneous expression and production of multiple antibodies and therapeutic proteins in vivo, and compared to other therapeutics, resulted in a significant decrease in administration frequency, and have a robust immunotherapeutic duration.

In general, plasmid transfer technology has traditionally been limited in scope because in vivo expression levels resulting from the naked DNA transfer have been low, only fractions of that achieved by viral gene transfer. Some investigators have outlined the safety and toxicological concerns with injecting viruses as DNA vectors into animals and humans (Pilaro and Serabian, 1999). Consequently, direct injection of plasmid DNA has become more attractive as a viable alternative. Persistent plasmid DNA transfer is accomplished with the application of a series of electric pulses to drive the DNA into a stable, non-dividing, population of cells. Skeletal muscle cells have provided an ideal target for direct plasmid transfer for DNA vaccines and other applications. Enhancement of plasmid delivery using electroporation allows the injected muscle to be used as a bioreactor for the persistent production and secretion of proteins into the blood stream. The expression levels are increased by as much as two to three orders of magnitude over plasmid injection alone, to levels comparable to those of adenoviral-mediated gene delivery and may in some cases reach physiological ranges.

The method of plasmid delivery in vivo, termed electroporation, electro-permeabilization, or electrokinetic enhancement, is simple, efficient and reproducible. It has become valuable for basic research, with great potential for gene transfer and DNA vaccination. Electroporation has been used very successfully to transfect tumor cells after injection of plasmid or to deliver the anti-tumor drug bleomycin to cutaneous and subcutaneous tumors in humans. Electroporation has been extensively used in mice, rats, dogs and pigs to deliver therapeutic genes that encode for a variety of hormones, cytokines, enzymes or antigens. The numerous tissues and organs that have been targeted include liver, skin, eye, testis, cardiac muscle, smooth muscle, tumors at different locations, and skeletal muscle.

Broadly, electroporation is the use of a transmembrane electric field pulse to induce microscopic pathways (pores) in a bio-membrane. These pores are commonly called “electropores.” Their presence allows macromolecules, ions, and water to pass from one side of the membrane to the other. Thus, electroporation has been used to introduce drugs, DNA or other molecules into multi-cellular tissues, and may prove to be effective for the treatment of certain diseases. However, the use of electroporation in living organisms has several problems, including cell death that results from generated heat and the inability of electropores to reseal. The beneficial effects of the drug or macromolecule are extremely limited with prior art electroporation methods where excessive cell heating and cell death occurs.

Several equations are helpful in understanding the process of electroporation. When a potential difference (voltage) is applied across the electrodes implanted in a tissue, it generates an electric field (“E”), which is the applied voltage (“V”) divided by the distance (“d”) between the electrodes (E=V/d).

The electric field intensity E has been a very important value in prior art when formulating electroporation protocols for the delivery of a drug or macromolecule into the cell of the subject. The field intensity is inversely proportional to the distance between the electrodes in that given a voltage, the field strength increases as the distance between the electrodes is decreased. However, a caveat is that an electric field can be generated in a tissue with insulated electrodes (i.e. flow of ions is not necessary to create an electric field). Without wishing to be bound by theory, it is the flow of ions that opens the electropores and allows movement of molecules into the cells of a subject during electroporation. The flow of electric charge in a conductor or medium between two points having a difference in potential is called the current. The current between electrodes is achieved by the ions or charged particles in the tissues, which can vary among tissues and patients. Furthermore, the flow of conducting ions in the tissue can change between electrodes from the beginning of the electric pulse to the end of the electric pulse.

When tissues have a small proportion of conducting ions, resistance is increased, heat is generated and cells are killed. Ohm's law expresses the relationship between current (“I”), voltage (“V”), and resistance (“R”) (R=V/I).

The resistance in the tissue between two electrodes can vary depending on the charged particles present therein. Thus, the resistance in the tissue changes from the beginning of the electric pulse to the end of the electric pulse.

Heating is the product of the inter-electrode impedance (i.e. combination of resistance and reactance and is measured in ohms), and is proportional to the product of the current, voltage and pulse duration. Heating can also be expressed as the square of the current, and pulse duration (“t”, time). For example, during electroporation the heating or power (“W”, watts) generated in the supporting tissue can be represented by the following equation: W=I² Rt

Broadly, metallic electrodes are sometimes placed in contact with tissues and short pulses of predetermined voltages are imposed on the electrodes initiating the cells to transiently open membrane pores. The protocols currently described for electroporation are defined in terms of the resulting field intensities E, which are dependent on short pulses of voltage proportional to the distance between the electrodes, and regardless of current. Accordingly, the resistance or heating cannot be determined for the electroporated tissue, which leads to varied success with different pulsed voltage electroporation protocols. Certainly, the difference in upper limit amplitudes of a voltage pulse between electroporation protocols that facilitate effective electroporation and electroporation protocols that cause the cells to die are very small. Additionally, a definite correlation has been observed between death of cells and the heating of cells caused by the upper limit amplitudes of the short voltage pulses. Thus, the over heating of cells between across electrodes serves as a principal cause for the ineffectiveness of any given electroporation voltage pulsing protocol. Furthermore, the current between electrodes serves as a primary determinant of the effectiveness of any given pulsing protocol, not the voltage across the electrodes.

When electricity is delivered to the cells of a subject, the dose of electricity can be accurately described in terms of charge (“Q”), which is the current (“I”) and the time (“t”), according to the formula: Q=It

If the current is not constant, as is the case in previously described electroporators, Q represents the time integral of I. In this respect, charged particles, be they ions or molecules, behave in a similar fashion. For example, when silver ions are deposited on an electrode to define the standard unit of electrical charge (the coulomb), only the charge, as defined above, is of importance. A certain minimum voltage must be present to generate a current, but the quantity of ions deposited can not be determined from a pre-determined voltage.

Correspondingly, the quantity of charged particles delivered to cells in an electroporator can not be derived from the voltage imposed on the electrodes.

In embodiments, the term “current” as used herein refers to the flow or rate of flow of electric charge in a conductor or medium between two points having a difference in potential, generally expressed in amperes.

In embodiments, the “ampere” as used herein refers to the standard unit for measuring the strength of an electric current. It is the rate of flow of charge in a conductor or conducting medium of one coulomb per second.

In embodiments, the “coulomb” as used herein refers to the meter-kilogram-second unit of electric charge equal in magnitude to the charge of 6.28×10¹⁸ electrons or the charge transported through a conductor by a current of one ampere flowing for one second.

In embodiments, the “voltage” as used herein refers to the electromotive force, or difference in electrical potential, expressed in volts, which are the practical units of electromotive force or difference in potential between two points in an electric field that requires one joule of work to move a positive charge of one coulomb from the point of lower potential to the point of higher potential.

In embodiments, the “power” as used herein refers to a source of physical or mechanical force or energy that is at, or can be put to, work, e.g. “electric power, water power.”

In embodiments, the “impedance” as used herein refers to the total opposition offered by an electric circuit to the flow of an alternating current of a single frequency. It is a combination of resistance and reactance and is measured in ohms.

In embodiments, the “field” as used herein refers to physical quantity specified at points throughout a region of space.

In embodiments, the term “amplitude” as used herein refers to the extreme range of a fluctuating quantity, as an alternating current or the swing of a pendulum, generally measured from the average or mean to the extreme. It is the amount or degree to which a thing extends.

In embodiments, the “frequency” as used herein refers to the number of periodic oscillations, vibrations, or waves per unit of time. It is usually expressed in hertz (Hz).

In some embodiments, one or more subcomponents of the device disclosed herein is described in U.S. Pat. No. 8,209,006, which is hereby incorporated by reference in its entirety.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising.” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein. All publications, patents, and patent publications cited are incorporated by reference herein in their entirety for all purposes.

This disclosure is further illustrated by the following non-limiting examples.

### EXAMPLES

### Example 1: Design And Application of Gene Transfer Device

In the experiments of this example, a variety of gene transfer devices were designed and applied to the delivery of a plasmid DNA construct through short electrical pulses. In these experiments, the gene transfer device was designed to feature an electrode array design and electrical pulsing parameters that are suggested to be suboptimal by published literature. Further, in this the example, the gene transfer device was shown to deliver the plasmid DNA construct to the muscle cell, which allowed for the simultaneous expression and production of multiple antibodies and therapeutic proteins in vivo, and compared to other therapeutics, resulted in a significant decrease in administration frequency, and have a robust immunotherapeutic duration.

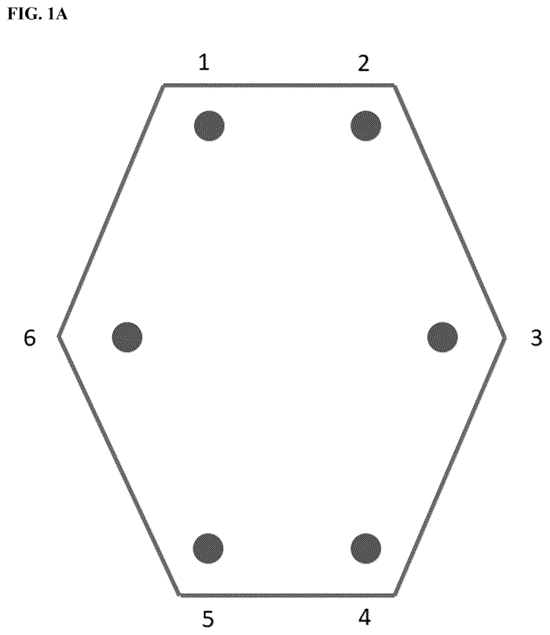

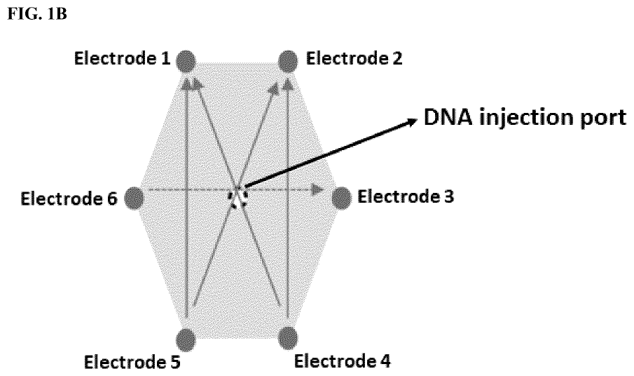

FIG. 1A and FIG. 1B are images showing the electrode and injection configuration of the gene transfer device, also referred to as the “barrel device” herein. The solid dark and solid green circles represent an electrode, and the clear circle represents a DNA injection port. Red arrows indicate the pulsing direction.

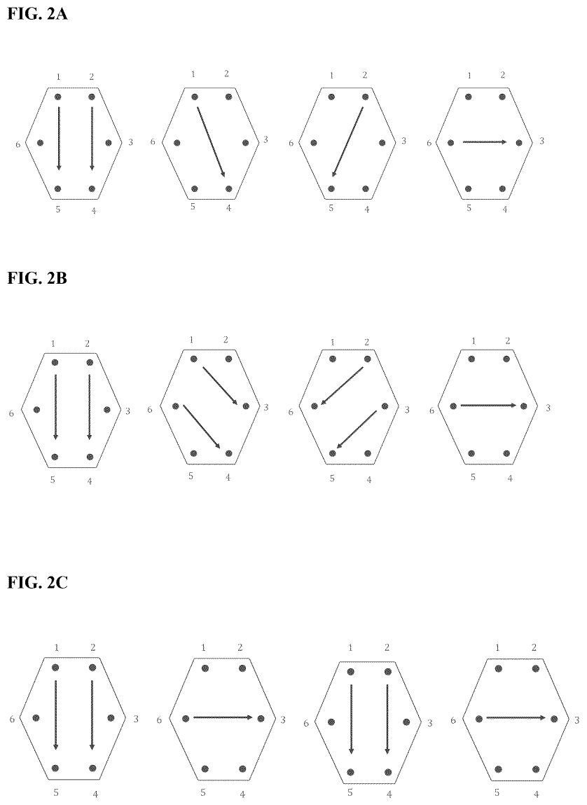

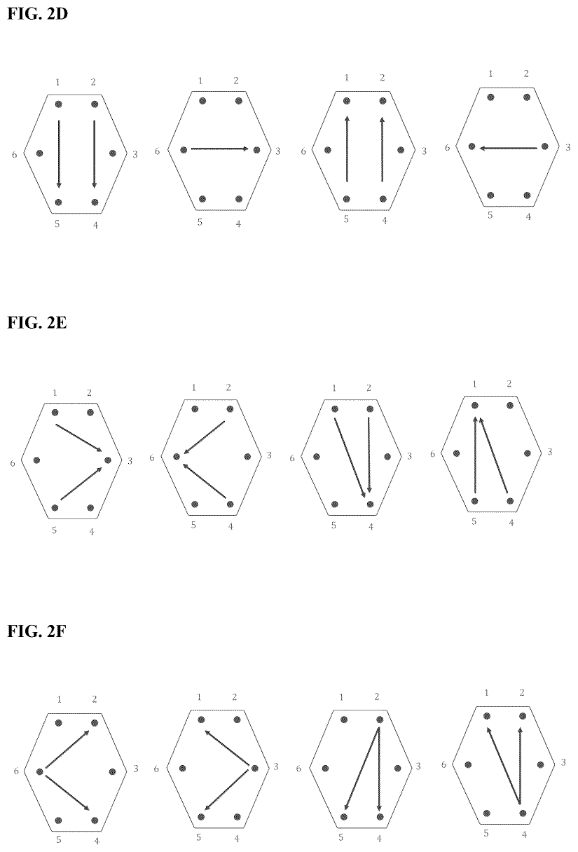

FIG. 2A, FIG. 2B, FIG. 2C, FIG. 2D, FIG. 2E, FIG. 2F, FIG. 2G, and FIG. 2H are images showing the configuration of electrode pulsing patterns. Red arrows indicate the pulsing direction.

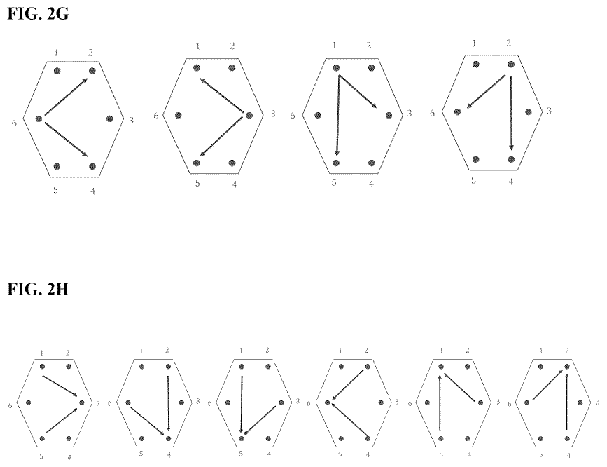

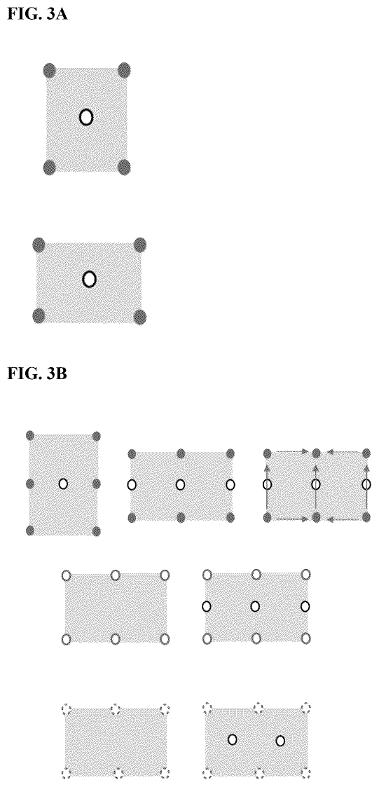

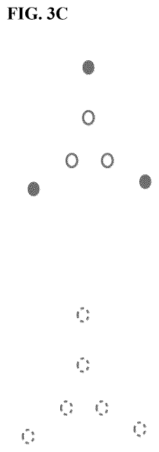

FIG. 3A, FIG. 3B, and FIG. 3C are images showing the configuration of electrode pulsing patterns. The solid dark and solid green circles represent an electrode, and the clear circle represents a DNA injection port. The dashed line clear circle represents an electrode with a DNA injection site at a side port. FIG. 3A (“4n device”) shows a device with four electrodes, FIG. 3B (“6n device”) shows a device with 6 electrodes, and FIG. 3C shows a device with electrodes in a star-type configuration (“star device”). In some embodiments, the electrode array may include a first electrode, a second electrode, a third electrode, a fourth electrode, a fifth electrode, and a sixth electrode. The electrodes may be arranged sequentially around the DNA injection port. The electrodes may each be positioned and/or arranged circumferentially from the DNA injection port. Each electrode may be equally spaced from respective neighboring electrodes of the electrode array. In some embodiments, the first electrode, the second electrode, the third electrode, the fourth electrode, the fifth electrode, and the sixth electrode may refer to any of electrode 1, electrode 2, electrode 3, electrode 4, electrode 5, and electrode 6.

In some embodiments, the electric pulses have a pulse pattern in the range of 1 MHz to 1,000 KHz. In some embodiments, the pulse pattern has at least 100, or at least 200, or at least 300, or at least 400, or at least 500, or at least 1000, or at least 2,500, or at least 5000 pulses, or at least 10,000 pulses, or at least 20,000 pulses, or at least 50,000 pulses for each burst.

The first electrode may neighbor the second electrode and the sixth electrode. The first electrode and the second electrode may be positioned along an axis that is parallel to the sixth electrode and the third electrode. The first electrode and the fourth electrode may be positioned along an axis that extends through the DNA injection port. The first electrode and the fifth electrode may be positioned along an axis that bisects an axis between the sixth electrode and the DNA injection port.

In some embodiments, the device disclosed herein provides directional stimulation that cause an array of electrodes to emit electric fields in the targeted tissue to maximize expression of a plasmid DNA construct. In some embodiments, a respective electric pulse generated by the pulse generator travels at an initial time period, and from any direction, from an initial electrode selected from the first, second, third, fourth, fifth, and/or sixth electrodes to a target electrode that is different from the selected initial electrode (i.e., see, without limitation FIGS. 2A, 2B, 2C, 2D, 2E, 2G, and 2H). In some embodiments, and at a second time period, a respective electric pulse generated by the pulse generator travels from the initial electrode to a second electrode, not selected from the initial time period, to generate an electric field that travels from the initial electrode to the second electrode. In some embodiments, the direction is vertical. In some embodiments, the direction is horizontal. In some embodiments, the direction is diagonal (i.e., see, without limitation, FIGS. 2A, 2B, 2C, 2D, 2E, 2G, and 2H). In some embodiments, the initial time of the pulse generation and the second time of the pulse generation are the same. In some embodiments, the initial time of the pulse generation and the second time of the pulse generation are different.

In some embodiments, a different number of electrodes are present in the device disclosed herein. For example, the device, in some embodiments, comprises at least 1 or more, at least 2 or more, at least 3 or more, at least 4 or more, at least 5 or more, at least 6 or more, at least 7 or more, at least 8 or more, at least 9 or more, at least 10 or more, at least 12 or more, at least 13 or more, at least 14 or more, or at least 15 or more electrodes.

In some embodiments, and while two time periods are described herein, additional time periods where different directional electric field patterns are implemented may also be included in other embodiments.

In some embodiments, a respective electric pulse generated by the pulse generator travels from the first electrode to at least one of the third, fourth, and/or fifth electrodes. In some embodiments, a respective electric pulse generated by the pulse generator travels from the first electrode to at least two of the third, fourth, and/or fifth electrodes. In some embodiments, the direction of travel is vertical. In some embodiments, the direction of travel is horizontal. In some embodiments, the direction of travel is diagonal (i.e., see, without limitation, FIGS. 2A, 2B, 2C, 2D, 2E, 2G, and 2H).

In some embodiments, a respective electric pulse generated by the pulse generator travels from the second electrode to at least one of the fourth, fifth, and/or sixth electrodes. In some embodiments, a respective electric pulse generated by the pulse generator travels from the second electrode to at least two of the fourth, fifth and/or sixth electrodes. In some embodiments, the direction of travel is vertical. In some embodiments, the direction of travel is horizontal. In some embodiments, the direction of travel is diagonal (i.e., see, without limitation, FIGS. 2A, 2B, 2C, 2D, 2E, 2G, and 2H).

In some embodiments, a respective electric pulse generated by the pulse generator travels from the third electrode to at least one of the first, fifth, and/or sixth electrodes. In some embodiments, a respective electric pulse generated by the pulse generator travels from the third electrode to at least two of the first, fifth and/or sixth electrodes. In some embodiments, the direction of travel is horizontal. In some embodiments, the direction of travel is diagonal (i.e., see, without limitation, FIGS. 2A, 2B, 2C. 2D, 2E, 2G, and 2H).

In some embodiments, a respective electric pulse generated by the pulse generator travels from the fourth electrode to at least one of the first, sixth, and/or second electrodes. In some embodiments, a respective electric pulse generated by the pulse generator travels from the fourth electrode to at least two of the first, fifth and/or sixth electrodes. In some embodiments, the direction of travel is horizontal. In some embodiments, the direction of travel is diagonal (i.e., see, without limitation, FIGS. 2A, 2B, 2C. 2D, 2E, 2G, and 2H).

In some embodiments, a respective electric pulse generated by the pulse generator travels from the fifth electrode to at least one of the first, second, and/or third electrodes. In some embodiments, a respective electric pulse generated by the pulse generator travels from the fifth electrode to at least two of the first, second, and/or third electrodes. In some embodiments, the direction of travel is horizontal. In some embodiments, the direction of travel is diagonal (i.e., see, without limitation, FIGS. 2A, 2B, 2C, 2D, 2E, 2G, and 2H).

In some embodiments, a respective electric pulse generated by the pulse generator travels from the sixth electrode to at least one of the second, third, and/or fourth electrodes. In some embodiments, a respective electric pulse generated by the pulse generator travels from the sixth electrode to at least two of the second, third, and/or fourth electrodes. In some embodiments, the direction of travel is horizontal. In some embodiments, the direction of travel is diagonal (i.e., see, without limitation, FIGS. 2A, 2B, 2C, 2D, 2E, 2G, and 2H).

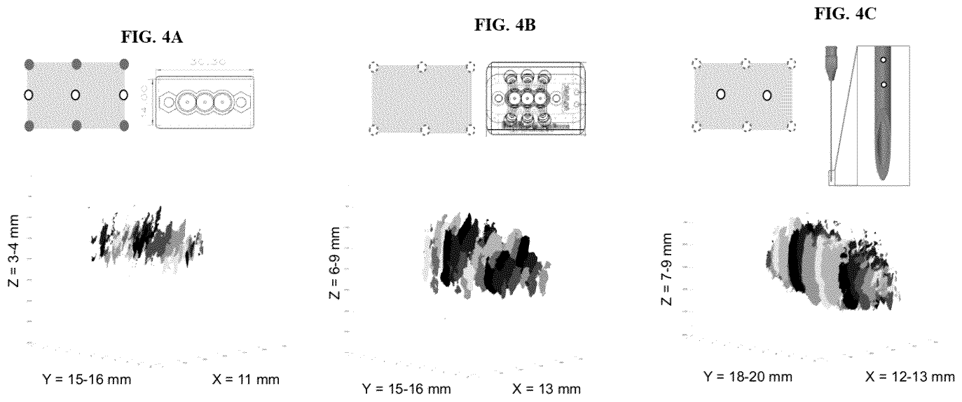

FIG. 4A, FIG. 4B, and FIG. 4C are images and graphs of the gene transfer device showing enhanced gene expression based on the configuration of the 6n device.

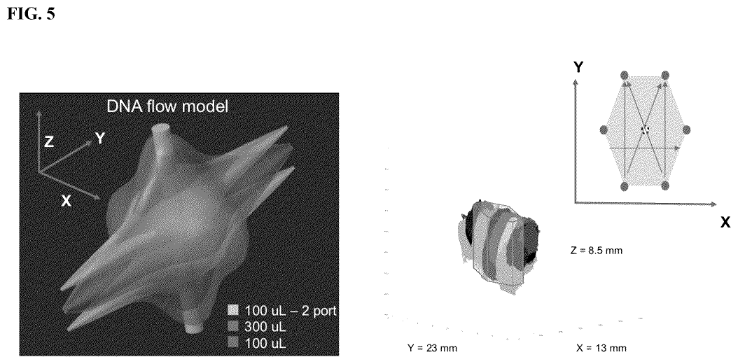

FIG. 5 are images and graphs of the gene transfer device showing enhanced gene expression based on the barrel device and the configuration of electrode pulsing patterns in FIG. 1A and FIG. 1B.

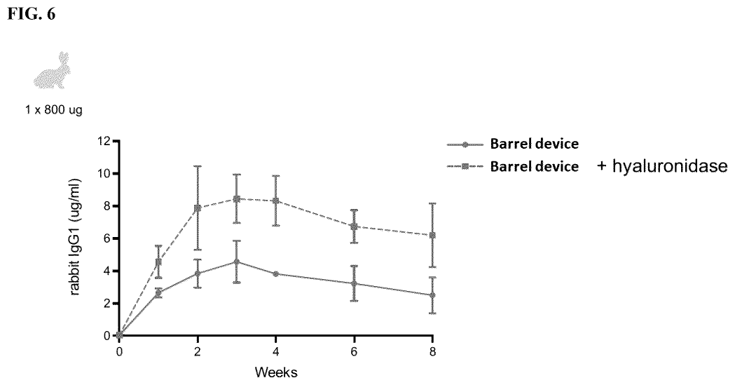

FIG. 6 is a graph showing the barrel device increasing the level of IgG1 in rabbits over a time course of several weeks.

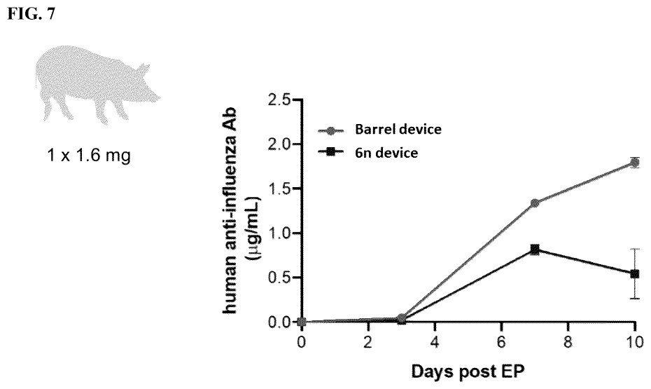

FIG. 7 is a graph showing the barrel device and 6n device increasing the level of human anti-influenza antibody levels in pigs.

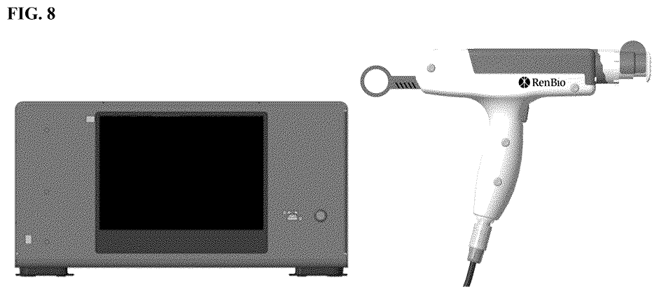

FIG. 8 is an image showing a first-in-human (FIH) generator (left) and handpiece (right) used in a phase 1 clinical trial.

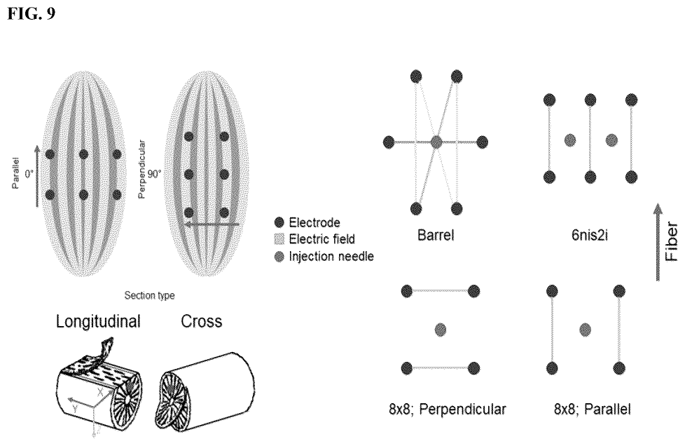

FIG. 9 shows images of the gene transfer devices disclosed herein. The top left image represents the orientation of a device relative to the fiber direction of a muscle. The bottom left image represents the tissue sectioning planes (used for imaging) as well as the directional axis used. The right half image represent four (4) devices and their respective electrode layout and pulsing scheme. The various shades on barrel indicate novel pairings/pulses of the electrodes.

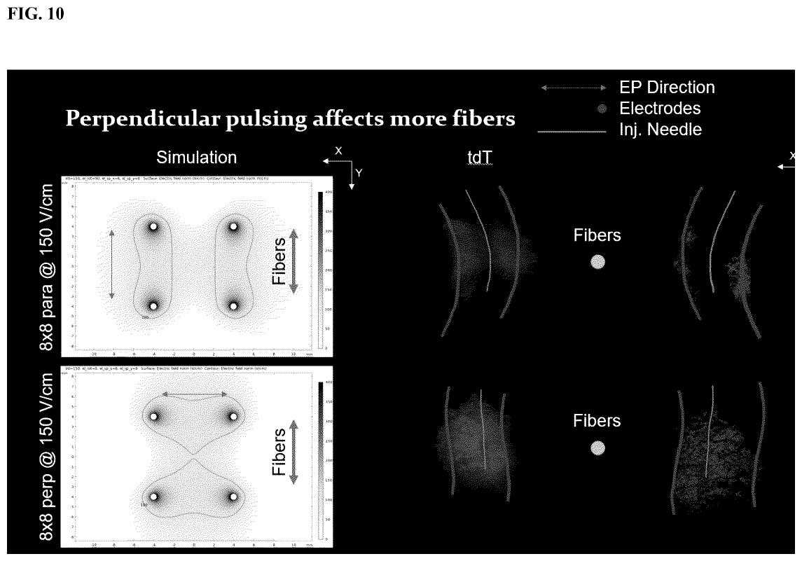

FIG. 10 shows images demonstrating perpendicular pulsing affects more muscle fibers. As shown in FIG. 10, electric fields follow along the paths of least resistance. As shown in FIG. 9, muscle are comprised of numerous elongated fibers. These fibers, when cross-sectioned, can be modeled as a series of capacitive shells. Furthermore, due to the natural formation of the fibers, the primary direction of fluid flow is along the fibers (in the Y axis) in the interstitial spaces between cells.

There are two consequences of this. First, the flow and conductance of the fluid may influence the electric field, and the inherent conductance. Second, the orientation of the fibers, relative to the field (parallel/perpendicular), influences how many cells are electroporated.

Parallel electroporation (“EP”) affects fibers which are localized to the electrodes. This is due to the muscle architecture. When electrodes start and end in the same fibers, as well as the same ECM pathways between said fiber lengths, there are no lesser resistive pathways to draw the current outwards to new cells. As such, only the few fibers between electrodes experience electroporation (EP). When it comes to perpendicular EP, no direct pathway exists between electrodes. As such the field is forced to spread out and saturate the space between electrodes. The images above on the right of FIG. 10 depict examples of parallel and perpendicular EP using the same device. DNA encoding for tdT (a fluorescent protein) was used. The red shown on the images represents the cells which were successfully electroporated.

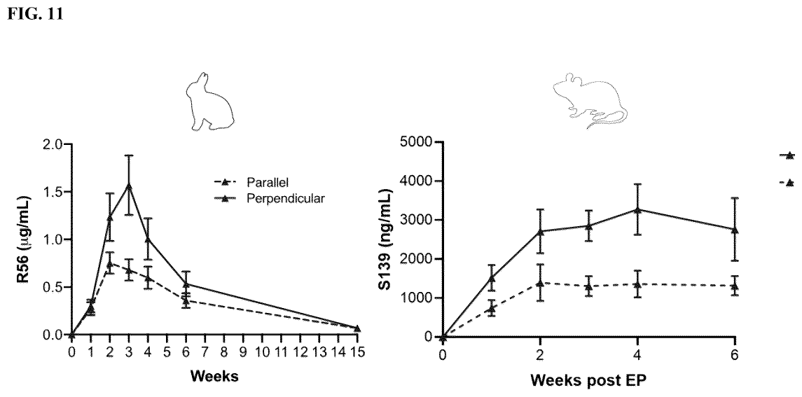

FIG. 11 shows graphs demonstrating how orientation affect electroporation efficiency. This figure shows how tdT (tdTomato) model predictions and imaging correlates with serum Ab levels observed. In the left graph of FIG. 11, a square 8×8 mm device was used in rabbits (4 electrode in a square pattern), which was simply rotated 90 degrees to orient the electric field either parallel to, or perpendicular to, the muscle fibers. A ˜2× fold increase in expression occurred, but the greater value achieved form perpendicular EP quickly fell off. In the right graph of FIG. 11, a similar experiment was performed in mice, confirming the benefit of orienting the electric field perpendicular to the muscle fibers.

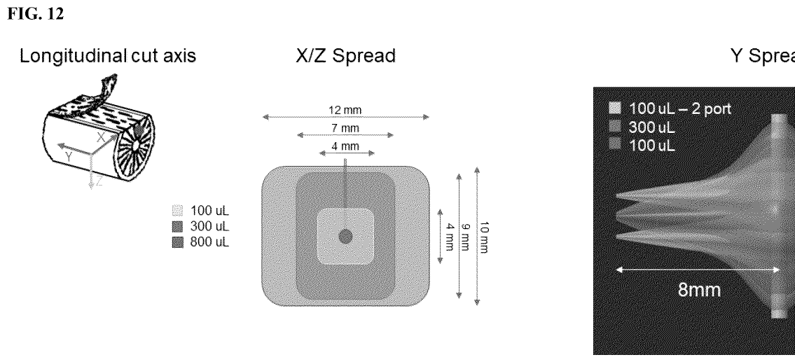

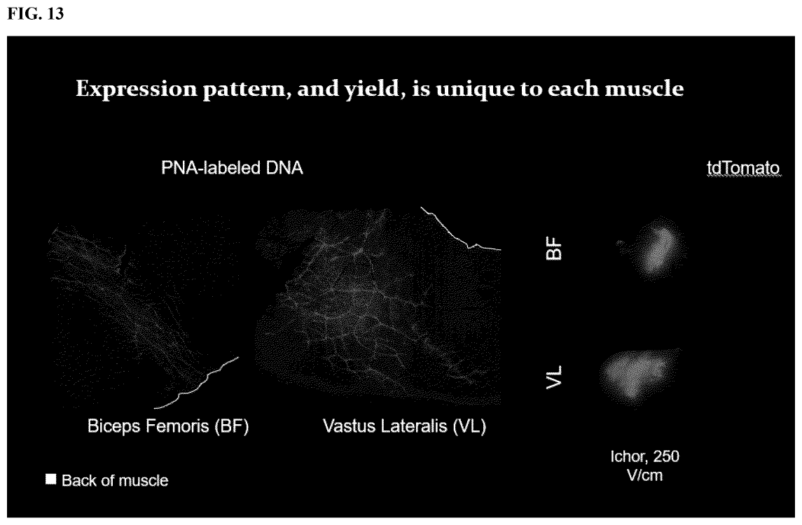

FIG. 12 shows images of a 2D and 3D representation collected and evaluated for the localization of the injected fluid containing the plasmid DNA. In this experiment, PNA-labeled plasmid DNA was used to show the spread of fluid in the muscle. This was then used to define the size and shape of the device. PNA is a reagent that fluorescently labels the plasmid DNA so it can be visualized after injection. PNA showed that DNA is present at least 8 mm away from the center in the Y axis (along the fibers) after injection. PNA also showed at least 12 mm of total spread in X axis at the injection site, tapering off to some distance beyond 8.

Prior studies showed that the shape of tdTomato expression was not filling the entire device footprint. The electric field was likely being wasted and causing excess damage to the area. As such, in this experiment, the shape/size of the device was dictated by the presence of the DNA being injected. To do so, a PNA reagent was used to label the plasmid DNA. The labeled DNA was injected, then the tissue was harvested/fixed/sectioned, and imaged on a microscope. These data were used to maximize the overlap of the electric field and plasmid DNA, thereby maximizing the efficiency of the device. One key discovery in this experiment was that DNA-containing fluid flow in the biceps differed from the vastus (the two main target muscles in the rabbit). The major axis of flow switch from the X in the vastus, to the Y and Z in the biceps. This discovery may change how the device is shaped in various environments.

FIG. 13 is an image showing the expression pattern and yield is unique to each muscle. The images on the left of FIG. 13 are from the indicated target muscle injected with PNA-labeled DNA. The images on the right of FIG. 13 are from tissue which has been injected with DNA encoding tdTomato fluorescent protein and electroporated.

As shown in both the sample sets, the fluid/DNA distribution patterns (PNA), and correlating expression (tdTomato), are unique for each muscle. This evaluation has helped guide selection in picking muscles and device types/dimensions for various therapies and targets. The tdT and PNA show significant diffusion (Z:X plane) patterns in the VL and stripped column-like (Z axis) patterns in the BF.

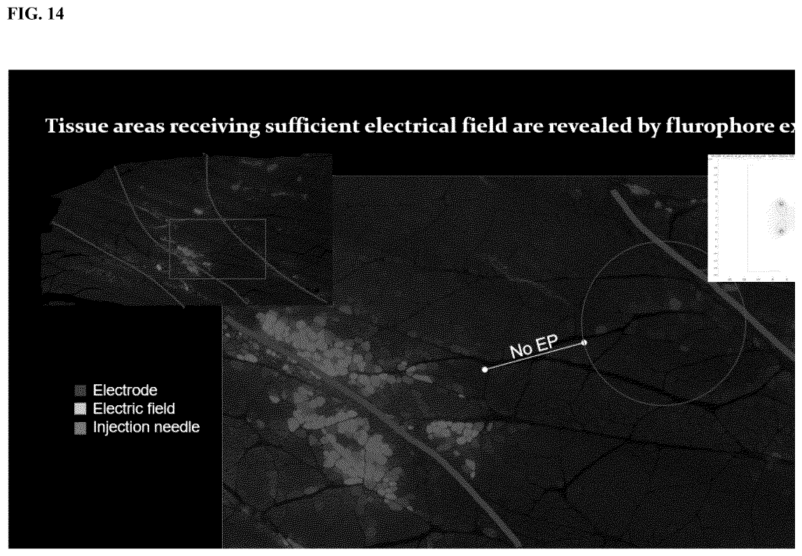

FIG. 14 is an image showing tissue areas receiving sufficient electrical field are revealed by fluorophore expression. The image in FIG. 14 is taken from a device in which the electrical field is pulsed in parallel pairs of electrodes (shown top right). The device used had three DNA injection sites, one between each pair of electrodes. In this study, a different fluorophore-expressing plasmid was injected at each site. This was done to identify where fluid from each injection was flowing. At the time, it was unknown that the field was insufficient between each pair of electrodes. In this experiment, as shown in the image above, it was demonstrated that fluid encoding for a red fluorescent protein showed up in two distinct areas (left and right side of the image of FIG. 14). The fluid encoding this could only come from a single place, meaning it had to have traveled through, and be present, in the area between the red fluorescence. The observation that there is a gap between these areas of red cells, means that the field strength was insufficient to electroporate these cells.

Using this information, the distance away from the electrodes that was above a sufficient field strength was determined. Going back to the model, the values were examined for the field at that distance away from the electrodes and determine the threshold of reversible electroporation.

Data from Mir et al (PNAS, 1999 pmid: 10200250) suggests that it is pulse duration which has a greater effect on the levels of electroporation, rather than the pulsing. In the past, it was understood that electrophoretic forces were generated by pulsing the field, causing charging and discharging of the particles. As the particles charged, they would migrate towards the expected pole.

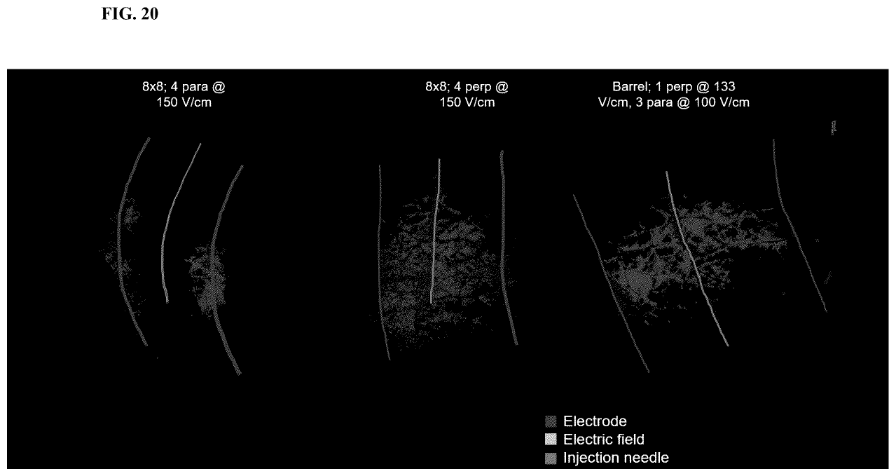

Even more surprising, is the observation that a quick (10 ms) pulse was able to create a significant portion of expression, indicative of successful electroporation. Previously data showed that this would result in no expression. These data support the use of single pulses in each orientation with the barrel device (FIG. 20).

Previous studies indicate that several pulses are needed. Thus, in accordance with published data, there is a significant (few fold to ˜ten-fold) drop in electroporation efficiency that occurs with decreasing the number of pulses. Accordingly, the gene transfer device disclosed herein (e.g., the barrel device) is a significant advance.

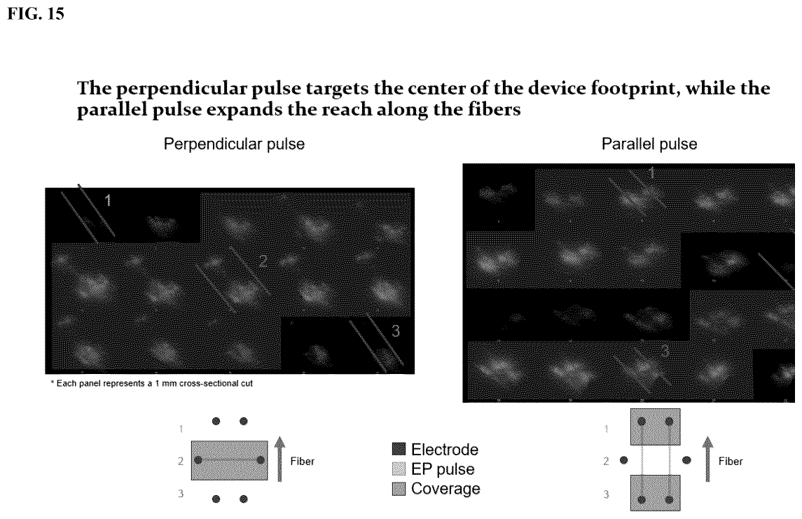

FIG. 15 is an image showing the perpendicular pulse targets the center of the device footprint, while the parallel pulse expands the reach along the fibers. As a corollary the pulses in parallel show focused concentrations of tdT fluorescence around the external electrodes. This expression fades towards the middle of the device (where the perpendicular pair is, labeled “2”). A simple representation of expression and pulsing is shown at the bottom of FIG. 15. This helps illustrate the coverage of each pulse. According to the COMSOL evaluation of each of these pulse types, there is little to no expression present in either setup.

The gene transfer device data disclosed herein suggests perpendicular EP is achieved at a lower threshold of transmembrane potential (TMP) than anticipated.

The data disclosed herein refutes the published thresholds of reversible and irreversible electroporation. According to the data published, poration in the transverse direction should require a greater voltage to reach TMP. This is also shown by their evaluation of 800 Vcm in the perpendicular (2× the voltage needed for parallel) being needed to produce irreversible EP. (See Čorović, Selma, et al. “The Influence of Skeletal Muscle Anisotropy on Electroporation: In Vivo Study and Numerical Modeling.” Medical & Biological Engineering & Computing, vol. 48, no. 7, 2010, pp. 637-648.)

In contrast, it was determined that for the gene transfer devices disclosed herein, the threshold for reversible EP to be ˜80 V/cm in the perpendicular direction.

The data of the present disclosure shows that under the same voltage conditions (between parallel and perpendicular), a much greater expression using perpendicular pulses is achieved.

This may be due to needle electrodes vs plate electrodes being used.

Another factor likely not touched up in parallel vs perpendicular EP for protein production, is the observation that a perpendicular EP is likely to affect a greater number of cells, relative to parallel.

Shown below is a representation of how electric field would flow (simplified). When in parallel, the field stays within the fibers it is initiated in. But in perpendicular, the field must cross the bounds of every fiber within the devices box.

Published research suggests that parallel EP is more effective at longer pulse lengths and lower voltages. (See Dermol-Černe, Janja, et al. “Short Microsecond Pulses Achieve Homogeneous Electroporation of Elongated Biological Cells Irrespective of Their Orientation in Electric Field.” Nature News, Nature Publishing Group, 4 Jun. 2020, https://www.nature.com/articles/s41598-020-65830-3.)

Published research suggests perpendicular EP requires nearly 2× voltages applied to achieve similar level of EP volume. (See Čorović, Selma, et al. “The Influence of Skeletal Muscle Anisotropy on Electroporation: In Vivo Study and Numerical Modeling.” Medical & Biological Engineering & Computing, vol. 48, no. 7, 2010, pp. 637-648.)

Published research suggests that cells are more likely to be porated in the parallel direction at the pulse duration disclosed herein (10 ms) (See Dermol-Černe, Janja, et al. “Short Microsecond Pulses Achieve Homogeneous Electroporation of Elongated Biological Cells Irrespective of Their Orientation in Electric Field.” Nature News, Nature Publishing Group, 4 Jun. 2020.).

Published research suggested that as the aspect ratio increased (longer cell axis length in parallel orientation), greater poration occurred due to parallel field EP (See Dermol-Černe, Janja, et al. “Short Microsecond Pulses Achieve Homogeneous Electroporation of Elongated Biological Cells Irrespective of Their Orientation in Electric Field.” Nature News, Nature Publishing Group, 4 Jun. 2020.).

According to published equations, the greatest TMP will be reached in the cell face that is in-line with the electric field. Induced transmembrane voltage/potential (Δϕm) or TMP is defined by the Schwan equation. The equation typically refers to spheres and not oblong spheroids, and even less so to spheroids with greatly discrepant axis lengths (such as a skeletal muscle cell).

Two versions from various papers are presented below:

$$Δ {ψ}_{membr} = 1.5 a {E}_{appl} cos θ \qquad [ 2 ]$$

$$\begin{matrix}The external field E is included as a condition on {Φ}_{e} . \\ {Φ}_{e} ( t , ρ , θ ) = - E ρ cos θ as ρ \to ∞ ,\end{matrix} \qquad ( 2 )$$

$$where ρ is the distance from the center of the cell and θ is the polar angle measured with respect to the direction of the field E . The current density is continuous across the cell membrane . \qquad [ 3 ]$$

This equation is taken from research based around spherical (or spheroid) cells. Most papers reference some version of this (or a time-domain version for modeling). One interesting note is the observation with “spheroids” with a 400× aspect ratio (suggesting the equation may no longer apply). See Krassowska, Wanda, and Petar D. Filev. “Modeling Electroporation in a Single Cell.” Biophysical Journal, vol. 92, no. 2, 2007, pp. 404-417., https://doi.org/10.1529/biophysj.106.094235. This version of the equation implies that the greatest TMP is experienced at the furthest point, in-line, with the field. Which in the case of parallel vs perpendicular is 400× in the parallel orientation. This theory coupled with the proceeding statement implies that TMP is uniform across the membrane. Meaning that it should theoretically be easiest to EP a cell by targeting its major axis.

Surprisingly, it was determined there is an equivalent voltage requirement for EP in either direction. Furthermore, this experiment resulted in more expression in when the EP was applied in the perpendicular orientation. This result may be due to DNA availability and location of poration.

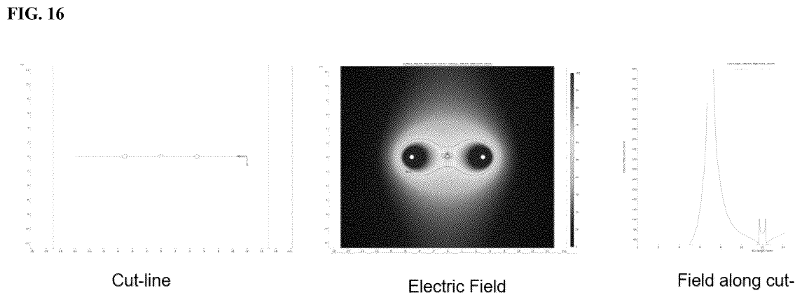

FIG. 16 is a graph and image showing how the side-port edge effect spikes the current locally. The image is a COMSOL evaluation of electric fields' effect on an elongated object present between the field. The object shown in the center can be considered infinitely long in the Z (into the page), while it has a finite r in either X/Y. This can be approximated as a fiber of r˜=40 um and a length (Z) of ˜12-40 mm. It is observed that the model predicts a decrease in TMP in the direction of EP and increase transverse to the field. This evaluation would predict that a parallel EP is best for fiber poration (as it would porate on all sides).

Theoretically this direction of poration should not be conducive for migration of DNA into the cell. This suggests, in conjunction with a single pulse being sufficient, that DNA may be present on the surface of the entire cell after injection and distribution, and that poration may need to be achieved somewhere on the surface. In other words, poration may be the strongest factor of EP and extra-cellular DNA migration may play a lesser/insignificant role.

Published research suggests that high frequency pulses are more capable of reaching and holding cells at TMP; while the devices disclosed herein data suggest low frequencies are capable of fully porating cells within the device footprint. See Murovec, Tomo, et al. “Modeling of Transmembrane Potential in Realistic Multicellular Structures before Electroporation.” Biophysical Journal, vol. 111, no. 10, 2016, pp. 2286-2295. The data in Murovec et al applies to “IRE” or irreversible EP. However, it can be extrapolated to imply poration or effective TMP being reached at respective parameters, but earlier. Research has typically shown that a higher frequency should bypass the capacitance of a cell to better penetrate muscle fascicles and provide a more uniform field. While this may likely be true to some extent, the data disclosed herein has shown that a very low frequency (1 Hz) is capable of near maximum reversible electroporation within the device footprint.

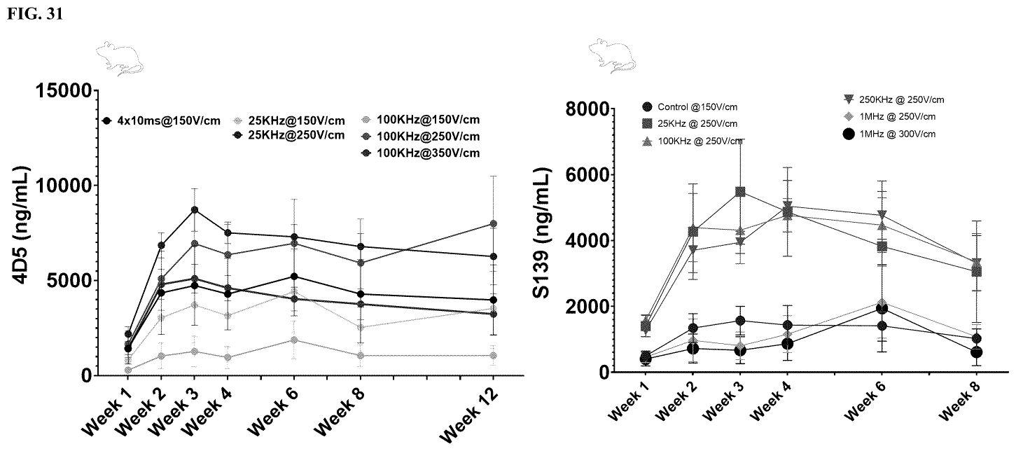

Furthermore, FIG. 31 displays the use of high frequency pulses at higher electric field strength yields better protein production compared to ms-wide pulses. In some embodiments, high pulses re applied in 4 bursts with each burst separated by 200 ms. In some embodiments, each burst has an ON time of 10 ms. Thus, in some embodiments, the 25 KHz pulse pattern has 500 pulses in each burst, the 100 KHz pulse pattern has 2000 pulses in each burst, the 250 KHz has 5000 pulses in each burst and the 1 MHz pulse has 20000 pulses in each burst.

In some embodiments, increasing frequency increases the electric field strength in order to induce pore formation and ultimately protein production. In some embodiments, 25 KHZ and 100 KHz frequency pulses are performed at 150 V/cm and yield low expression levels. In some embodiments, 250 V/cm exhibits significant increases in protein expression levels.

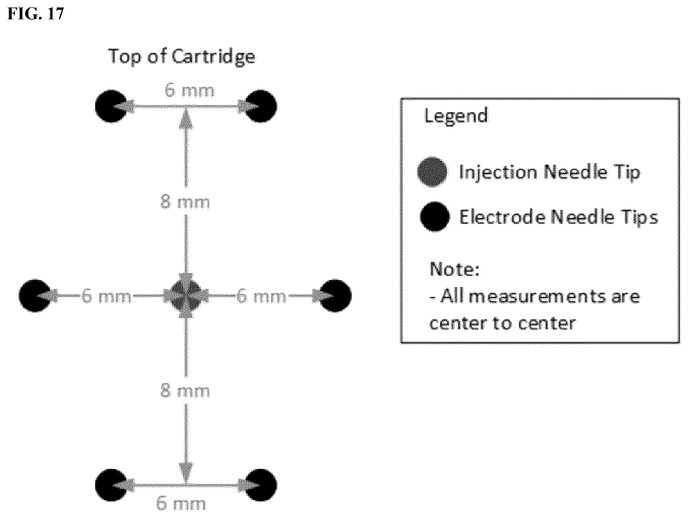

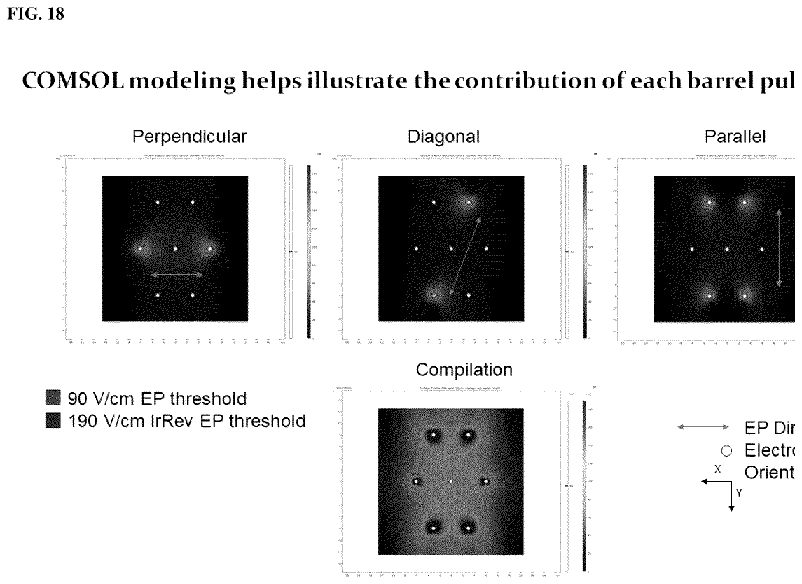

FIG. 17 is an image of the barrel device design configuration. The shape of the Barrel (FIG. 9) and on the right) was dictated by the following fluid spread measurements in the vastus lateralis muscle: for a single center injection of 800 uL, the DNA spreads: 12 mm in the X, 10 mm in the Z, and 8 mm in the Y. Shown in FIG. 17 is the final product of fluid flow and spread analysis. The major axis here is the Y direction, which is along the fiber, and was designed with the vastus muscle in mind. The minor axis is the X direction and represents the perpendicular direction. This set of 6 electrodes was designed to offer the best coverage of the maximum amount of fluid. The electrode array and pulsing pattern (FIG. 18) were also designed to offer the potential to mitigate any off-angle placements of the device. 800 uL was chosen as an optimum injected volume as it was capable of saturating a large area and penetrating within fascicle bundles, not just following the perimysium. The “pairing” (positive and negative poles) of the electrodes is shown on the next slide.

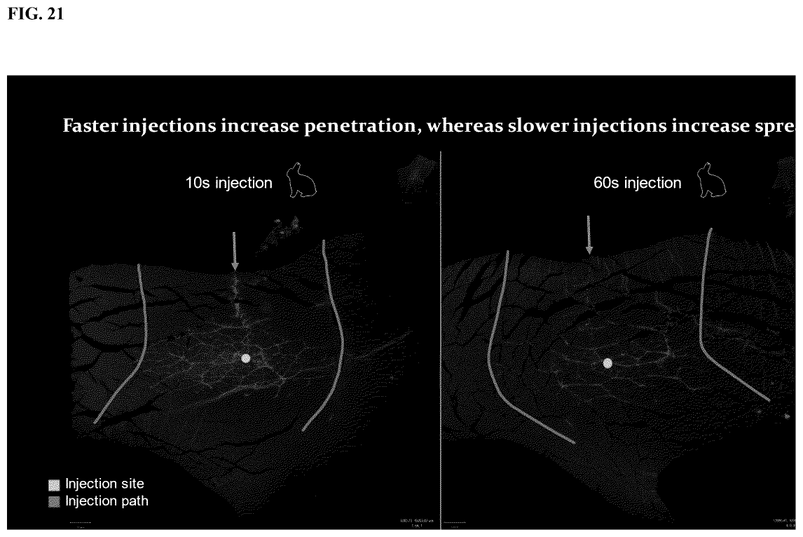

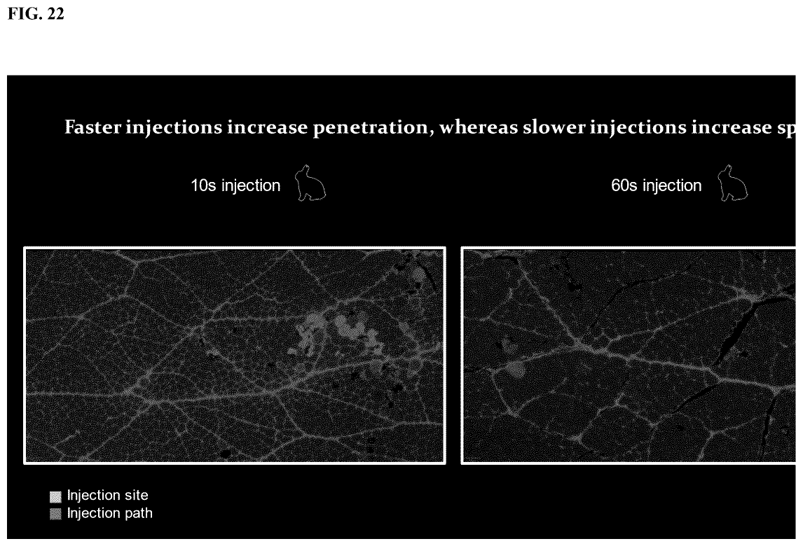

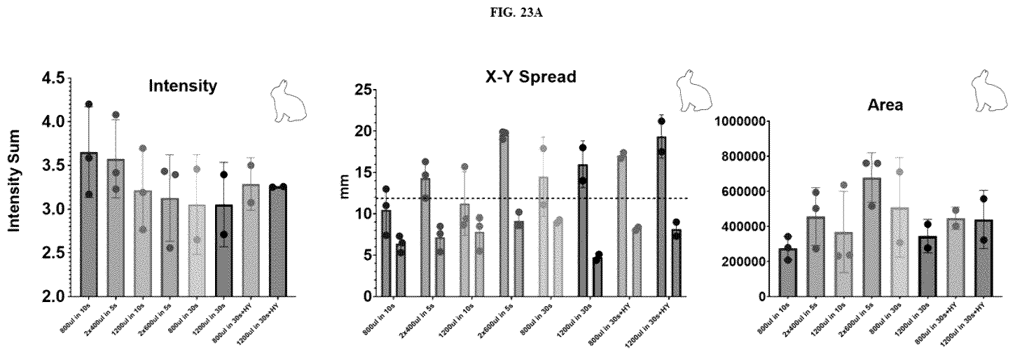

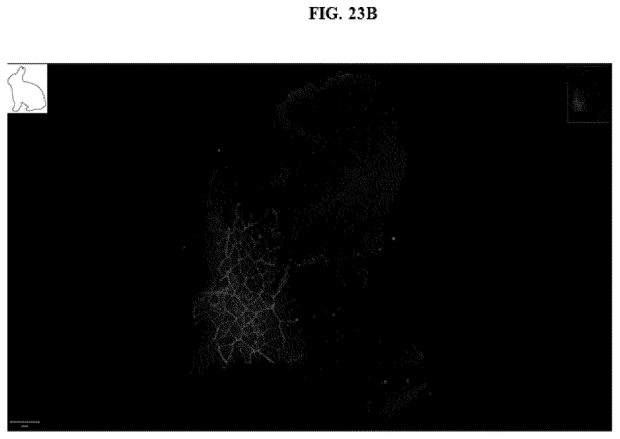

FIG. 21 and FIG. 22 are images showing PNA labeling and fluorescent imaging. These experiments were performed to visualize how physical parameters effect the behavior of fluid. In the case of injection rate, 800 uL was injected over a range of 10 to 60 seconds. Both the fastest and slowest rate illustrated a trade-off between fluid penetration and overall diffusion. In these experiments, the fastest injection enabled the fluid to more uniformly fill and penetrate fascicles while being more localized to the injection site. The slowest injection rate was more capable of diffusing further from the injection site. Both effects are illustrated in FIG. 21 and FIG. 22 (where FIG. 22 shows a higher magnification of the images shown in FIG. 21). The trade off between spread and penetration can be balanced to enhance protein expression by controlling the injection rate, ensuring that the fluid adequately saturates the cells, while staying within the device bounds.

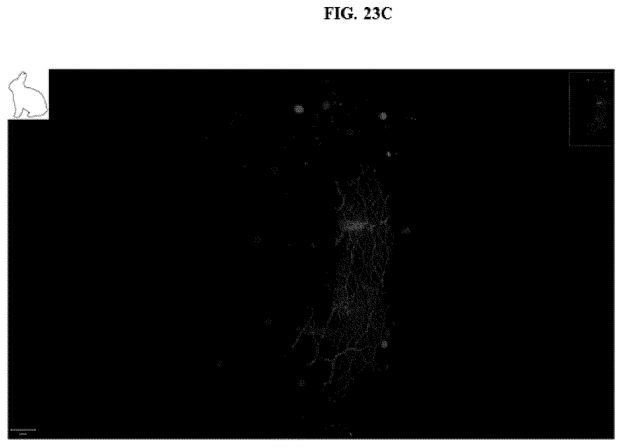

Surprisingly, injecting 800 uL using a single injection point over 30 s yielded a better fluid spread in the muscle when compared to injecting it over 10 s (FIG. 23A, FIG. 23B, FIG. 23C). Without wishing to be bound by theory, the results of these experiments led to 800 μL injected over 20 seconds being chosen as the final rate.

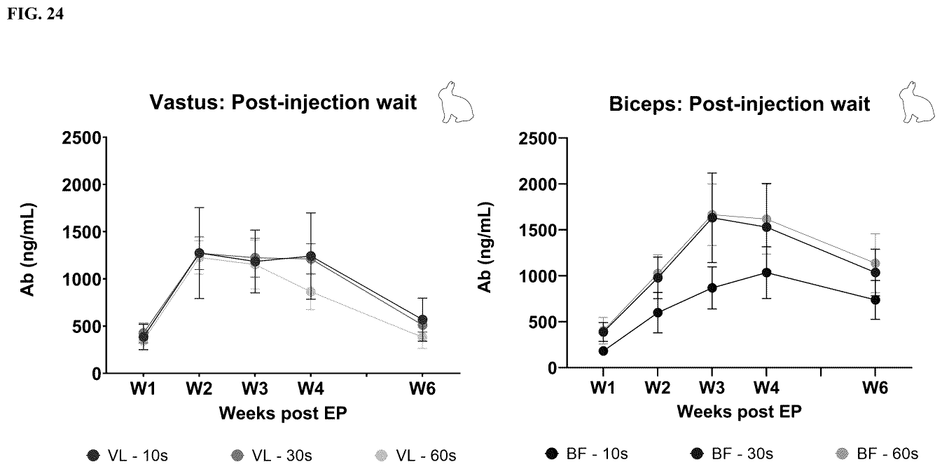

However, a fast injection (800 ul in 10 s) achieved better fascicle penetration of the fluid. Switching to dual injection of 400 uL, each-injected in 5 s (injection points separated by 5 mm, FIG. 23A, FIG. 23B, FIG. 23C), was shown to improve the X-Y spread of the injected DNA in the target area when compared to a single injection of 800 uL injected in 10 s, as well as achieved better fascicle penetration compared to a single injection of 800 uL in 30 s. The results of these experiments increased fascicle penetration while maintaining the distribution benefits of a slow injection. Similar to the effect injection time has, the length of time between the completion of the therapeutic protein injection and electroporation was shown in these experiments to have an impact on EP efficiency. In these experiments, post-injection wait time had a minor effect on protein expression from the vastus of a rabbit. However, when applied to the biceps of rabbits, expression was increased from previously inferior levels, as shown in the experiments of FIG. 24. This effect is due to the differences in muscle architecture.

Previous images show that a longer wait time provides better distribution of the plasmid, which may be responsible for the increase in protein expression.

FIG. 18 shows images of COMSOL modeling to illustrate the contribution of each barrel pulse. Perpendicular pulsing was incorporated to target as many fibers as possible across the largest area of fluid flow. A single pulse in perpendicular direction was used as other data suggested that too many pulses in the perpendicular orientation were damaging and potentially detrimental to expression.

The perpendicular pulse was supplemented with parallel and diagonal pulses. This serves to cover the entire shape of the fluid flow with sufficient electric field, while offering some amount of angle compensation. With the diagonal pulses, as the device is rotated, one will become more perpendicular and the other more parallel. The stacking of individual pulses helps mitigate damage in areas by evenly distributing the field, reducing the chances of irreversible EP from occurring.

A rudimentary model has been provided above to elaborate how the culmination of every pulse serves to efficiently electroporate the entire area of the barrel device.

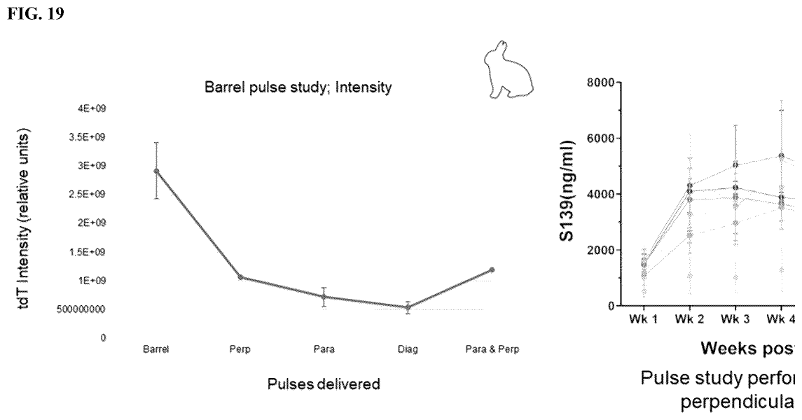

The black line represents the threshold for electroporation, while the dark red is meant to show areas likely to experience damage (closest to the electrodes). Experimental validation of this concept is shown on FIG. 19.

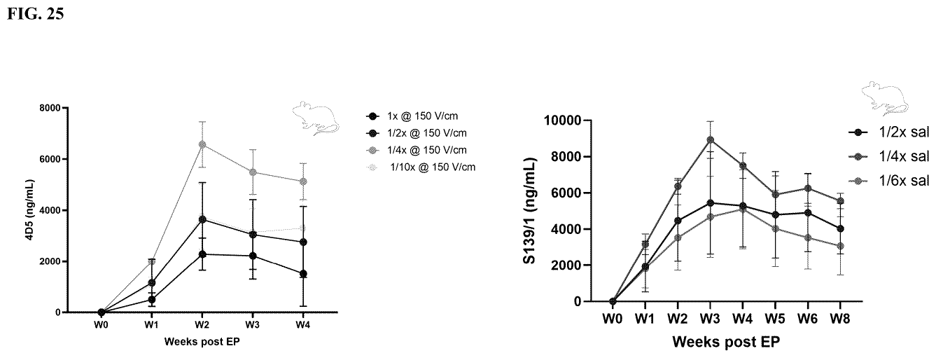

The boundaries and shape of the model significantly change with adjustments to the conductance of the medium. The experiments in FIG. 25 show how various solutions can be used to control the flow of the electric field in the target tissue. Conductivity of the injected fluid is an often overlooked and ignored component of electroporation. Most published studies use standard fluid bases of either PBS or 1× saline.

The experiments disclosed herein show that conductivity, relative to the conductivity of the target tissue, has a significant role in guiding the PEF (pulsed electric field). When the conductivity of the fluid is >2× higher than the conductivity of the tissue, decreases are observed in expression. These are observations are due to the presence of fluid in the ECM (extracellular matrix) in the path of least resistance, and the PEF follows along the outside of the cells, instead of passing through the cells. This leads to a reduction in the chances of reaching the TMP (trans-membrane potential), and successful poration are reduced.

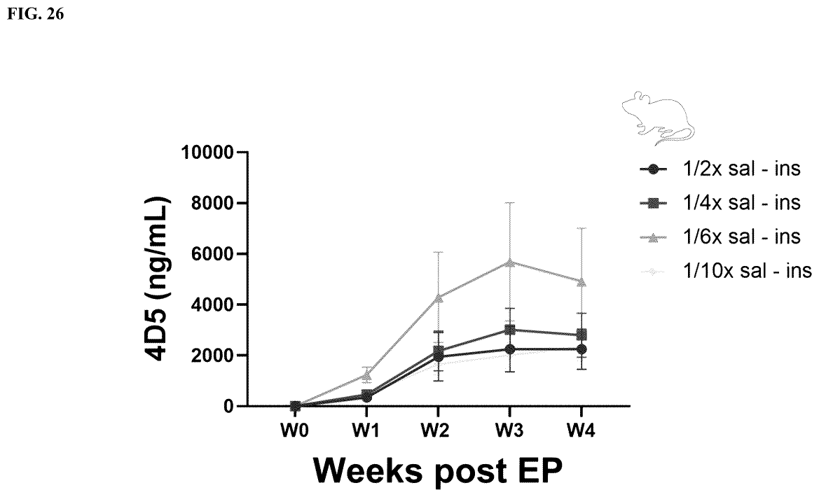

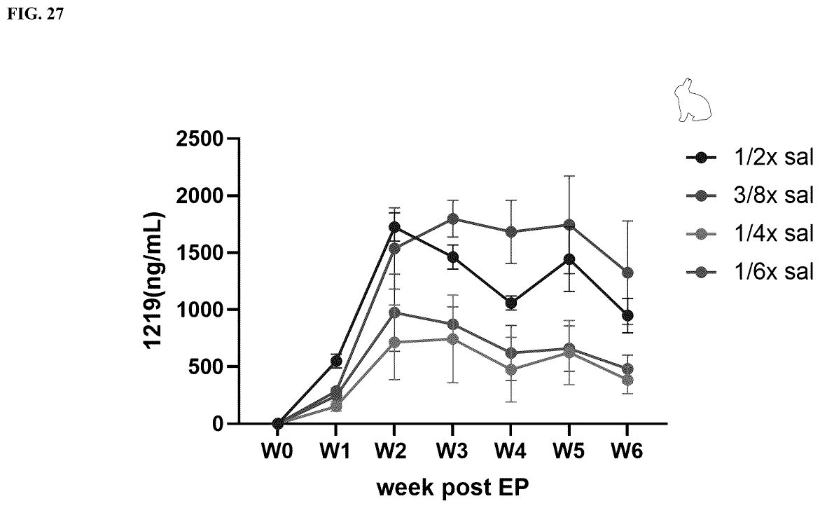

Further, the experiments disclosed herein show low conductivity has effects in guiding protein expression. As the conductivity approaches the target tissue, the entire target system becomes uniform in conductivity, which causes naturally lower resistant pathways to draw current from the target. This is shown in FIG. 25, FIG. 26, and FIG. 27. In each of FIG. 25, FIG. 26, and FIG. 27, there is a median conductivity, varying between species, which performs at a optimum level. Surprisingly, buffer conductivity must be tailored to each target tissue. In these experiments, it was observed that conductance of the DNA buffer must be marginally higher than the conductance of the target tissue and should be adjusted for each unique tissue. Furthermore, this means that an un-insulated electrode, touching the muscles membrane, or interstitial space, loses energy via those pathways, circumventing both the cells and the DNA.

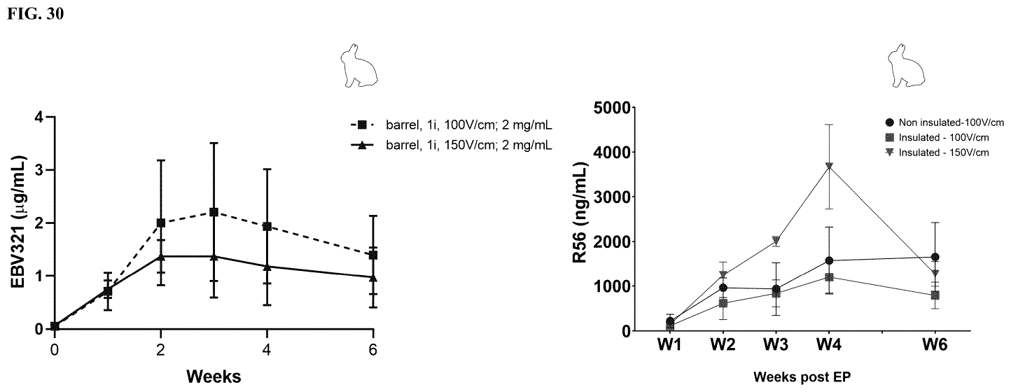

Without the use of insulation, it is necessary to design buffer conductivity containing the pDNA (plasmid DNA) to help ensure most of the electric field is being contained within the target muscle, instead of circumventing the target tissue via alternative pathways. FIG. 30 shows that by adding insulation, higher conductance pathways were eliminated, allowing the conductance of the fluid to be closer to that of the muscle, and resulting in higher expression levels. The closer the conductance of the fluid is to the tissue, the more uniform the flow of the electric field. The more uniform flow of the field allows for more electroporation of cells. and an enhancement in protein production. With a conductance closer to that of the target tissue itself, potential for pockets of fluid (of higher conductance) to draw the field away from cells are eliminated, thereby creating an effect like that of inserting a direct pathway between the two electrodes.

FIG. 30 shows how the addition of insulation to electrodes allows an increase in the voltage to be applied, thereby increasing expression. In the absence of adding insulation, a higher voltage results in a less desirable expression level (FIG. 30, left panel). This is due to high conductance pathways, which focuses the field, causing more damage at higher voltages. However, once those pathways are eliminated by adding insulation, increasing the voltage results in higher expression (FIG. 30, right panel).

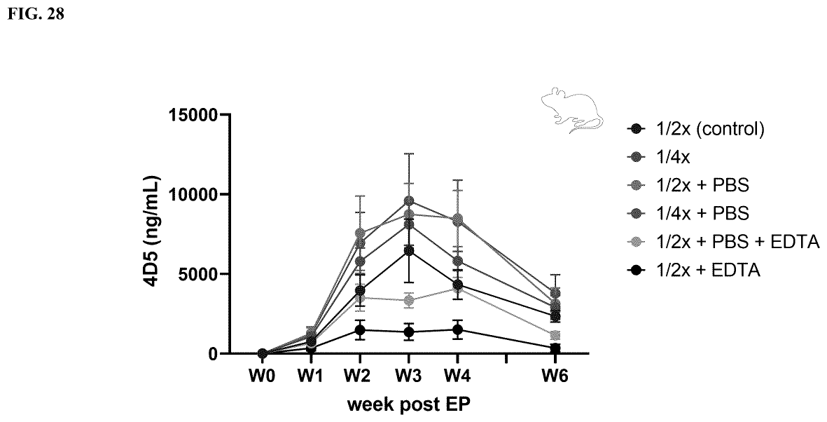

Industry standards of adding EDTA to DNA formulations is not a viable option for protecting DNA during storage (FIG. 28). The addition of EDTA was found to decrease protein production.

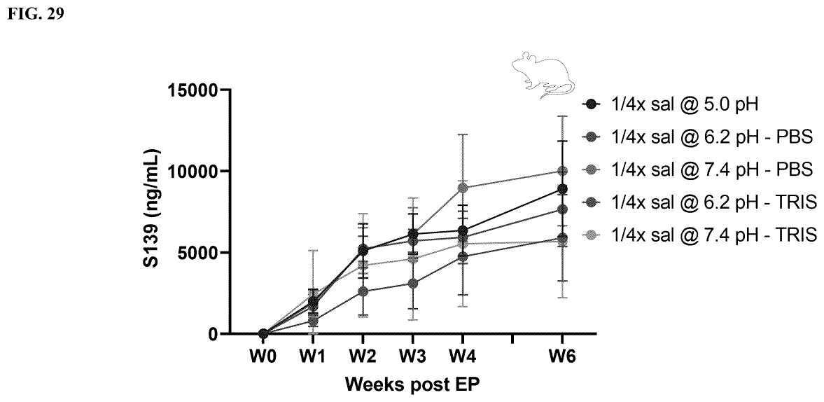

DNA being formulated at a certain pH can enhance or hinder efficient electroporation (FIG. 29). It is standard practice in the field to use 1× saline or phosphate-buffered saline (PBS) as a formulation for plasmid DNA when combined with electroporation. Saline has an unbuffered pH of 5.0. This pH can nick DNA, reducing its potency. PBS has a similar conductivity to 1× saline, though it is buffered to neutral pH. As described above, the experiments of this example have discovered that a lower conductivity, closer to the target tissue, is beneficial. The experiments of this example have also shown that a neutral pH is beneficial to the storage of DNA, as well as to the protein production levels when PBS is used as the buffer.

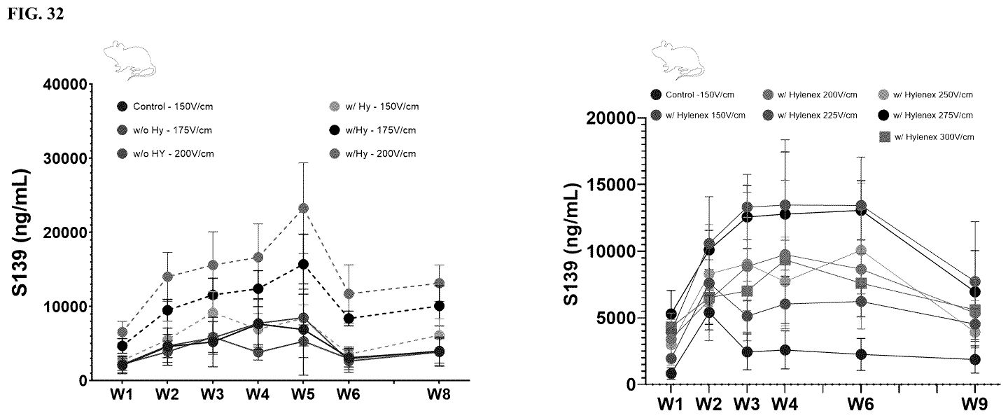

FIG. 32 exhibits the effect of voltage escalation of antibody expression when hyaluronidase is used. Hyaluronidase is known to breakdown the extracellular matrix and therefore allows for better spread of the pDNA in the target muscle. Increasing the applied electric field strength when using hyaluronidase in conjunction with pDNA has shown to improve protein production levels when compared to control electric field strength (FIG. 32).

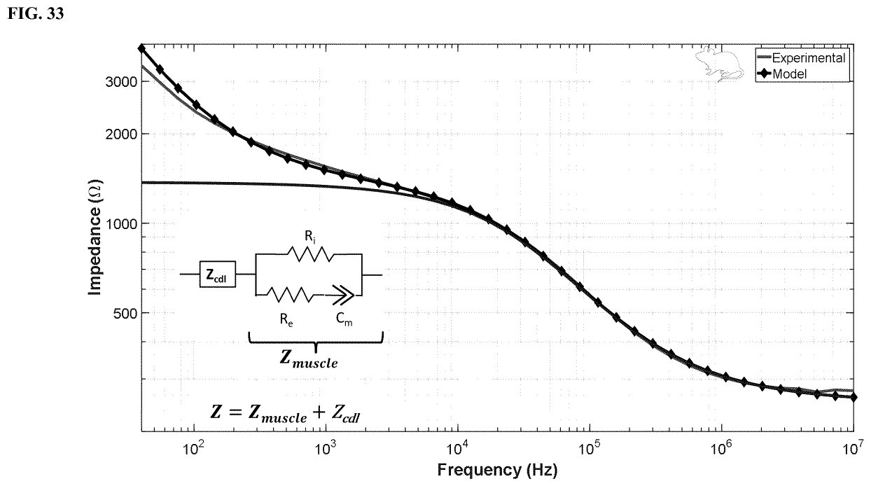

FIG. 33 portrays the impedance spectroscopy of a target muscle. The experimentally obtained impedance curve is exhibited by the red line. The muscle impedance modeled as a combination of resistor, capacitors and Constant Phase elements is represented by the blue line. The low frequency region of the impedance is dominated by Capacitive double layer (represented as Z_{cdl}) and not relevant to target muscle is also modelled using constant-phase elements. Modelling the muscle impedance +Z_{cdl} and fitting a curve obtained from the circuit portrayed (black line) gives parameters that convey information related to extracellular and intracellular fluid impedances as well as muscle cell capacitances.

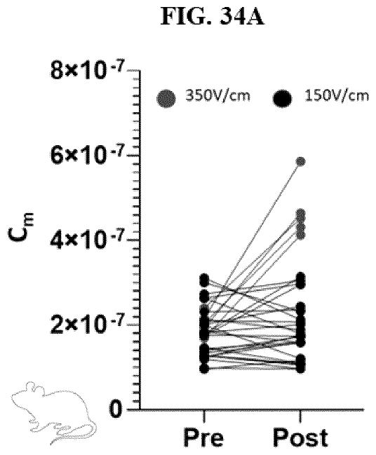

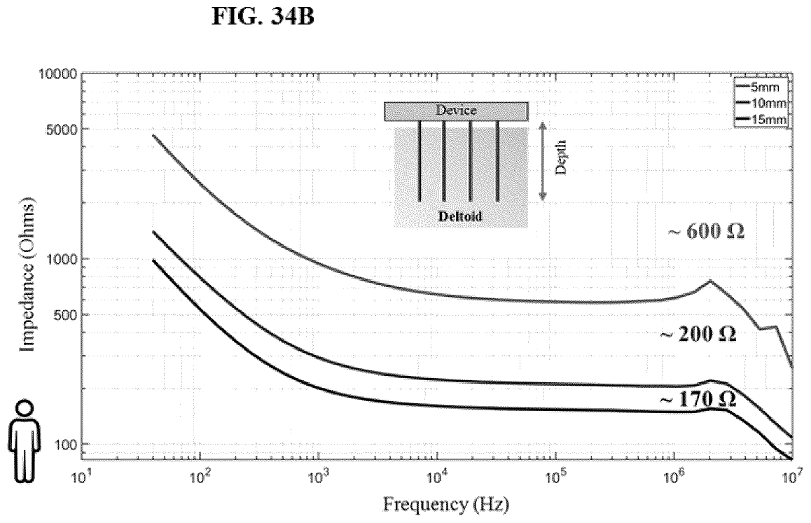

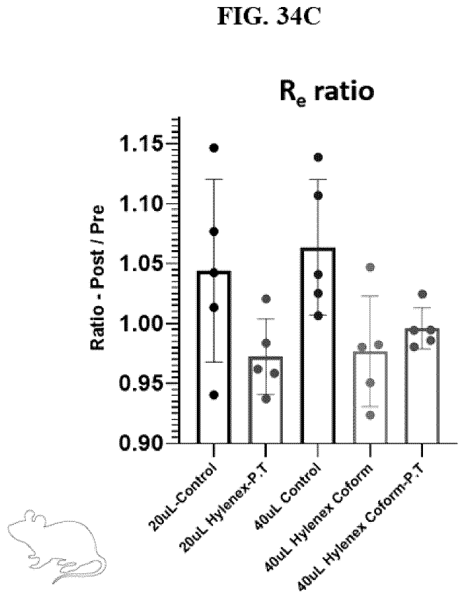

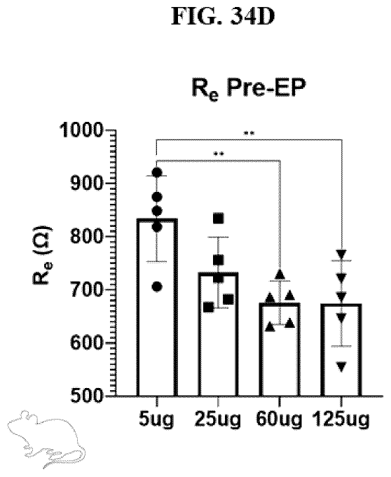

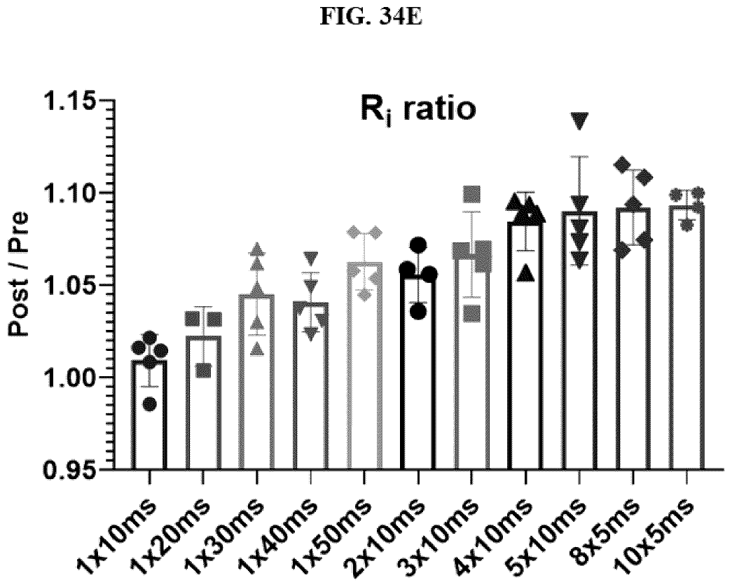

FIG. 34A, FIG. 34B, FIG. 34C, FIG. 34D, and FIG. 34E demonstrate the usefulness of impedance curve fitting and extracting parameters from the electrical circuit model. Changes in these parameters relate to different experimental conditions and are indicative of changes happening in the target muscle cells. FIG. 34A shows the change in muscle capacitance parameter when a very damaging field strength (350 V/cm) is applied, instead of control field strength (150 V/cm). FIG. 34B shows the magnitude of the impedance, indicating the muscle cells the electrodes have penetrated. Fat tissue has higher impedance compared to muscle cells, and therefore impedance magnitude values can be used to judge muscle depth. FIG. 34C shows the ratio of the Rₑ parameter (Post-EP Rₑ/Pre-EP Rₑ), which indicates a significant difference in the target muscle when Hylenex was used. FIG. 34D shows the parameter Rₑ also changes based on the concentration of pDNA injected (same volume). FIG. 34E shows the ratio of the Rᵢ parameter (Post Rᵢ/Pre Rᵢ) is indicative of the total energy received by the target muscle. Different types of pulses were used in these experiments, and the ratio of Rᵢ indicated an increase with increasing amount of energy delivered.

FIG. 19 are graphs demonstrating quantification of tdTomato showing the need for each pulse for maximal expression in barrel. The data shown on the left illustrates the additive benefit of each individual pulse. Various pulsing types were used (perpendicular and parallel) to mitigate damage while targeting as many fibers as possible. No single pulse type is responsible for the entirety of expression.

Data (right side of FIG. 19) have also shown that the addition of consecutive pulses is beneficial to the electroporation efficiency, in this case following delivery of a plasmid encoding an antibody (S139), instead of a fluorophore. The benefit of additional pulses appears to saturate around 3 pulses, which is important to know in order to keep the number of unique pulses as low as possible in order to mitigate patient discomfort and minimize tissue damage.

FIG. 20 is an image showing parallel vs perpendicular pulsing and the threshold for electroporation, and the how barrel device disclosed herein requires a lower electric field (V/cm) to successfully electroporate a large volume of muscle fibers. All three images have injection points central to the device. All injections are the same volume and DNA concentration.

As shown in FIG. 20, parallel EP fails to porate any fibers outside of the electrode pairs path (fibers and pairs are traveling into/out of the page). This couples with very minimal DAPI infiltration (blue staining of nuclei).

Perpendicular EP affects every fiber between the electrodes. Since the field and fibers are perpendicular to each other there is a greater intersection of the two. This can be seen in the center image where a greater area is expressing tdTomato. This area also is more restricted to the injection site. Unfortunately, with perpendicular pulsing of equivalent strength and duration as parallel, this experiment shows a greater presence of DAPI, indicative of immune cell infiltration and possible tissue damage.

Barrel, therefore, contains 1 perpendicular and 3 parallel (in various forms) pulses, in an attempt to extract the benefits of perpendicular pulsing, but relying on supplemental parallel pulses to target a maximal volume of tissue with minimal damage. With the combination of these two styles of pulsing, both a greater distribution of expression (not as dense as with purely perpendicular pulses) and a lesser extent of immune infiltration (though greater than with parallel pulsing alone) was obtained.

Together, these experiments demonstrate that, inter alia, a variety of gene transfer devices were designed and applied to the delivery of a plasmid DNA construct through short electrical pulses. In these experiments, the gene transfer device was designed to feature an electrode array design and electrical pulsing parameters that are suggested to be suboptimal by published literature. Further, in the experiments of this example, the gene transfer device was shown to deliver the plasmid DNA construct to the muscle cell, which allowed for the simultaneous expression and production of multiple antibodies and therapeutic proteins in vivo, and compared to other therapeutics, resulted in a significant decrease in administration frequency, and have a robust therapeutic duration.

All of the features disclosed herein may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

What is claimed is:

1. A device for gene transfer, the device comprising:
a handpiece;
an array of electrodes arranged at one end of the handpiece and configured to be positioned at a host cell of a subject; and
a pulse generator configured to generate electric pulses that cause the array of electrodes to emit electric fields in the targeted tissue to maximize expression of a plasmid DNA construct delivered therethrough while minimizing applied voltage and total electrical dose.

2. The device of claim 1, further comprising a DNA injection port configured to administer the plasmid DNA to the host cell, wherein the electrode array comprises a first electrode, a second electrode, a third electrode, a fourth electrode, a fifth electrode and a sixth electrode positioned circumferentially around the DNA injection port.

3. The device of claim 2, wherein a respective electric pulse generated by the pulse generator travels vertically or diagonally from the first electrode to at least one of the third, fourth, and/or fifth electrodes.

4. The device of any one of claims 2-3, wherein a respective electric pulse generated by the pulse generator travels vertically or diagonally from the first electrode to at least two of the third, fourth, and/or fifth electrodes.

5. The device of any one of claims 2-4, wherein a respective electric pulse generated by the pulse generator travels vertically or diagonally from the second electrode to at least one of the fourth, fifth, and/or sixth electrodes.

6. The device of any one of claims 2-5, wherein a respective electric pulse generated by the pulse generator travels vertically or diagonally from the second electrode to at least two of the fourth, fifth and/or sixth electrodes.

7. The device of any one of claims 2-6, wherein a respective electric pulse generated by the pulse generator travels vertically or diagonally from the third electrode to at least one of the first, fifth, and/or sixth electrodes.

8. The device of any one of claims 2-7, wherein a respective electric pulse generated by the pulse generator travels vertically or diagonally from the third electrode to at least two of the first, fifth and/or sixth electrodes.

9. The device of any one of claims 2-7, wherein a respective electric pulse generated by the pulse generator travels vertically or diagonally from the fourth electrode to at least one of the first, sixth, and/or second electrodes.

10. The device of any one of claims 2-9, wherein a respective electric pulse generated by the pulse generator travels vertically or diagonally from the fourth electrode to at least two of the first, fifth and/or sixth electrodes.

11. The device of any one of claims 2-10, wherein a respective electric pulse generated by the pulse generator travels vertically or diagonally from the fifth electrode to at least one of the first, second, and/or third electrodes.

12. The device of any one of claims 2-11, wherein a respective electric pulse generated by the pulse generator travels vertically or diagonally from the fifth electrode to at least two of the first, second, and/or third electrodes.

13. The device of any one of claims 2-12, wherein a respective electric pulse generated by the pulse generator travels vertically or diagonally from the sixth electrode to at least one of the second, third, and/or fourth electrodes.

14. The device of any one of claims 2-13, wherein a respective electric pulse generated by the pulse generator travels vertically or diagonally from the sixth electrode to at least two of the second, third, and/or fourth electrodes.

15. The device of any one of claims 2-14, wherein a respective electric pulse generated by the pulse generator travels from the fourth electrode to at least one of the first electrode and the second electrode.

16. The device of claim 2, wherein a respective electric pulse generated by the pulse generator travels vertically from the fourth electrode to the second electrode.

17. The device of any one of claims 2 and 16, wherein a respective electric pulse generated by the pulse generator travels diagonally from the fourth electrode to the first electrode.

18. The device of any one of claims 2, 16 and 17, wherein a respective electric pulse generated by the pulse generator travels from the fifth electrode to at least one of the first electrode and the second electrode.

19. The device of any one of claims 2, and 16-18, wherein a respective electric pulse generated by the pulse generator travels from the fifth electrode to the first electrode.

20. The device of any one of claims 2, and 16-19, wherein a respective electric pulse generated by the pulse generator travels diagonally from the fifth electrode to the second electrode.

21. The device of any one of claims 2, and 16-20, wherein a respective electric pulse generated by the pulse generator travels horizontally from the sixth electrode to the third electrode.

22. The device of any one of claims 1-21, wherein the pulse is a perpendicular pulse relative to the orientation of a muscle fiber.

23. The device of any one of claims 1-21, wherein the pulse is a parallel pulse relative to the orientation of a muscle fiber.

24. The device of any one of claims 1-23, wherein the device comprises an injection needle tip and an electrode needle tip having a distance of one of: at least 2 mm, at least 3 mm, at least 4 mm, at least 5 mm, at least 6 mm, at least 7 mm, at least 8 mm, at least 9 mm, at least 10 mm, at least 11 mm, at least 12 mm, at least 13 mm, at least 14 mm, at least 15 mm, at least 16 mm, at least 17 mm, at least 18 mm, at least 19 mm, or at least 20 mm between the injection needle tip and the electrode needle tip.

25. The device of any one of claims 1-24, wherein the electric pulses have a pulse pattern in the range of 1 MHz to 1,000 KHz.

26. The device of claim 25, wherein the pulse pattern has at least 100, or at least 200, or at least 300, or at least 400, or at least 500, or at least 1000, or at least 2,500, or at least 5000 pulses, or at least 10,000 pulses, or at least 20,000 pulses, or at least 50,000 pulses for each burst.

27. A method of delivering DNA to a subject, the method comprising:
a. loading the device of any one of claims 1-26 with a plasmid DNA construct encoding a therapeutic protein; and
b injecting the DNA plasmid into a host cell, thereby delivering the DNA to the subject.

28. The method of claim 27, wherein the host cell is a muscle cell.

29. The method of claim 27 or 28, wherein the DNA is injected both intramuscularly and in the extracellular space of the host cell.

30. The method of any one of claims 27-29, wherein the DNA is taken up in the host cell by electroporation.

31. The method of any one of claims 27-30, wherein the plasmid DNA construct is selected from SEQ ID NOs: 1-27.


SEQUENCE LISTING (27 sequences)

SEQ ID NO: 1 - DNA, 2259 bp, synthetic construct
  source 1..2259: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 2 - DNA, 52 bp, synthetic construct
  source 1..52: mol_type = other DNA
tcttttctgcagtcaccgtcgctagcctcgagagatcacttctggctaataa

SEQ ID NO: 3 - DNA, 1564 bp, synthetic construct
  source 1..1564: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatctcgttgctgccgcgcgcgccaccagacataatcgctgacacactgacagactgttcctttcctttttttttttttgcagtcaccgtcgctagcctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 4 - DNA, 52 bp, synthetic construct
  source 1..52: mol_type = other DNA
ttttttttgcagtcaccgtcgctagcctcgagagatcacttctggctaataa

SEQ ID NO: 5 - DNA, 2337 bp, synthetic construct
  source 1..2337: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcaggagaaagagaggatccagcggcgaaggcagaggcagccttcttacatgtggcgacgtggaagagaaccccggacctctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 6 - DNA, 46 bp, synthetic construct
  source 1..46: mol_type = other DNA
tcttttctgcagtcaccgtcgctagcaggagaaagagaggatccag

SEQ ID NO: 7 - DNA, 45 bp, synthetic construct
  source 1..45: mol_type = other DNA
ggaagagaaccccggacctctcgagagatcacttctggctaataa

SEQ ID NO: 8 - DNA, 2340 bp, synthetic construct
  source 1..2340: mol_type = other DNA
attggctattggccaactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgccgcggccgggaacggtgcattggaacgcggattccccgtgccaagagtgacgtaagtaccgcctatagactctataggcacacccctttggctcttatgcatgctatactgtttttggcttggggcctatacacccccgcttccttatgctataggtgatggtatagcttagcctataggtgtgggttattgaccattattgaccactcccctattggtgacgatactttccattactaatccataacatggctctttgccacaactatctctattggctatatgccaatactctgtccttcagagactgacacggactctgtatttttacaggatggggtcccatttattatttacaaattcacatatacaacaacgccgtcccccgtgcccgcagtttttattaaacatagcgtgggatctccacgcgaatctcgggtacgtgttccggacatgggctcttctccggtagcggcggagcttccacatccgagccctggtcccatgcctccagcggctcatggtcgctcggcagctccttgctcctaacagtggaggccagacttaggcacagcacaatgcccaccaccaccagtgtgccgcacaaggccgtggcggtagggtatgtgtctgaaaatgagcgtggagattgggctcgcacggctgacgcagatggaagacttaaggcagcggcagaagaagatgcaggcagctgagttgttgtattctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtcgctagcaggagaaagagaggatccagcggcgccaccaacttcagcctgcttaaacaggcaggcgacgtggaagagaaccccggacctctcgagagatcacttctggctaataaaagatcagagctctagagatctgtgtgttggttttttgtggatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcagcacagcaagggggaggattgggaagacaatagcaggcatgctggggatgcggtgggctctatgggtacctctctctctctctctctctctctctctctctctctctctcggtacctctctctctctctctctctctctctctctctctctctctcggtaccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaatt

SEQ ID NO: 9 - DNA, 46 bp, synthetic construct; identical to SEQ ID NO: 6
  source 1..46: mol_type = other DNA

SEQ ID NO: 10 - DNA, 46 bp, synthetic construct
  source 1..46: mol_type = other DNA
tggaagagaaccccggacctctcgagagatcacttctggctaataa

SEQ ID NO: 11 - protein, 466 aa, synthetic construct
  REGION 1..466: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 238 aa, synthetic construct
  REGION 1..238: note = Synthetic Sequence
MVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 730 aa, synthetic construct
  REGION 1..730: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 731 aa, synthetic construct
  REGION 1..731: note = Synthetic Sequence
MDWTWRVFCLLAVAPGAHSEVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGQSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 468 aa, synthetic construct
  REGION 1..468: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 235 aa, synthetic construct
  REGION 1..235: note = Synthetic Sequence
MVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 729 aa, synthetic construct
  REGION 1..729: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 730 aa, synthetic construct
  REGION 1..730: note = Synthetic Sequence
MDWTWRVFCLLAVAPGAHSEVQLVQSGAEVKKPGESLKISCKGSGYSFTTYWLGWVRQMPGKGLDWIGIMSPVDSDIRYSPSFQGQVTMSVDKSITTAYLQWNSLKASDTAMYYCARRRPGQGYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGDIQMTQSPSSLSASVGDRVTITCRASQGISSWLAWYQQKPEKAPKSLIYAASSLQSGVPSRFSGSGSGTDFTLTISSLQPEDFATYYCQQYNIYPYTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 469 aa, synthetic construct
  REGION 1..469: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 234 aa, synthetic construct
  REGION 1..234: note = Synthetic Sequence
MVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 729 aa, synthetic construct
  REGION 1..729: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGEGRGSLLTCGDVEENPGPMVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 730 aa, synthetic construct
  REGION 1..730: note = Synthetic Sequence
MDWTWRVFCLLAVAPGAHSQVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRRKRGSSGATNFSLLKQAGDVEENPGPMVLQTQVFISLLLWISGAYGDIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 204 aa, synthetic construct
  REGION 1..204: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 24 - protein, 226 aa, synthetic construct
  REGION 1..226: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSHHHHHHVSGWRLFKKIS

SEQ ID NO: 25 - protein, 390 aa, synthetic construct
  REGION 1..390: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGGSGGGGSGGGGSVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 26 - protein, 215 aa, synthetic construct
  REGION 1..215: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSHHHHHH

SEQ ID NO: 27 - protein, 220 aa, synthetic construct
  REGION 1..220: note = Synthetic Sequence
MAGPATQSPMKLMALQLLLWHSALWTVQEATPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQPGGGGSVSGWRLFKKIS